SEQUENCE LISTING (96 sequences)

SEQ ID NO: 1 - protein, 124 aa, Artificial Sequence
  Synthetic
EVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARSRYSSSLRRYYGMDVWGQGTTVTVSS

SEQ ID NO: 2 - protein, 9 aa, Artificial Sequence
  Synthetic
FTFSSYAMH

SEQ ID NO: 3 - protein, 14 aa, Artificial Sequence
  Synthetic
WVAVISYDGSNKYY

SEQ ID NO: 4 - protein, 16 aa, Artificial Sequence
  Synthetic
RSRYSSSLRRYYGMDV

SEQ ID NO: 5 - protein, 107 aa, Artificial Sequence
  Synthetic
AIQLTQSPSSLSASVGDRVTITCQATHDIANYLNWYQQKPGKAPKLLIYAASNLETGVPSRFSGSGSGTDFTFAISSLQPEDIAVYYCQQYDNLPLTFGGGTKVEIK

SEQ ID NO: 6 - protein, 8 aa, Artificial Sequence
  Synthetic
HDIANYLN

SEQ ID NO: 7 - protein, 11 aa, Artificial Sequence
  Synthetic
LLIYAASNLET

SEQ ID NO: 8 - protein, 8 aa, Artificial Sequence
  Synthetic
QQYDNLPL

SEQ ID NO: 9 - protein, 126 aa, Artificial Sequence
  Synthetic
QMQLVQSGAEVKKPGSSVKVSCKASGGTFSSYAISWVRQAPGQGLEWMGRIIPILGIANYAQKFQGRVTITADKSTSTAYMELSSLRSEDTAVYYCAVNTYYYDSSGYYYEYFDYWGQGTLVTVSS

SEQ ID NO: 10 - protein, 9 aa, Artificial Sequence
  Synthetic
GTFSSYAIS

SEQ ID NO: 11 - protein, 14 aa, Artificial Sequence
  Synthetic
WMGRIIPILGIANY

SEQ ID NO: 12 - protein, 18 aa, Artificial Sequence
  Synthetic
VNTYYYDSSGYYYEYFDY

SEQ ID NO: 13 - protein, 107 aa, Artificial Sequence
  Synthetic
EIVLTQSPSSLSASVGDRVTITCQASQHINNHLNWYQQKPGKGPKLLIYDVSNLEAGVPSRFSASGSGTDFSFTISSLQPEDFATYYCQQYDSLPLTFGQGTKVEVK

SEQ ID NO: 14 - protein, 8 aa, Artificial Sequence
  Synthetic
QHINNHLN

SEQ ID NO: 15 - protein, 11 aa, Artificial Sequence
  Synthetic
LLIYDVSNLEA

SEQ ID NO: 16 - protein, 8 aa, Artificial Sequence
  Synthetic
QQYDSLPL

SEQ ID NO: 17 - protein, 128 aa, Artificial Sequence
  Synthetic
QMQLVQSGGGVVQPGRSLRLSCAASGFTFSSYGMHWVRQAPGKGLEWVAVISYDGSNKYYADSVKDRFTISRDNSKNTLYLQMNSLRAEDTAVYYCAKDIVGDSGSYPIYYYYGMDVWGQGTTVTVSS

SEQ ID NO: 18 - protein, 9 aa, Artificial Sequence
  Synthetic
FTFSSYGMH

SEQ ID NO: 19 - protein, 14 aa, Artificial Sequence
  Synthetic
WVAVISYDGSNKYY

SEQ ID NO: 20 - protein, 20 aa, Artificial Sequence
  Synthetic
KDIVGDSGSYPIYYYYGMDV

SEQ ID NO: 21 - protein, 111 aa, Artificial Sequence
  Synthetic
SQSALTQPHSVSESPGKTVTISCTRSSGSIASNYVQWYQQRPGSAPSTVIYEDNQRPSGVPDRFSGSVDISSNSASLTISGLQTEDEADYYCQSYDNDNMVFGGGTKLTVL

SEQ ID NO: 22 - protein, 10 aa, Artificial Sequence
  Synthetic
SGSIASNYVQ

SEQ ID NO: 23 - protein, 12 aa, Artificial Sequence
  Synthetic
STVIYEDNQRPS

SEQ ID NO: 24 - protein, 8 aa, Artificial Sequence
  Synthetic
QSYDNDNM

SEQ ID NO: 25 - protein, 126 aa, Artificial Sequence
  Synthetic
EVQLVESGGGLEKPGRSLRLSCTASGFTFGDYAMSWFRQAPGKGLEWVGFITTKPYGGTTEYAASVKGRFTISRDDSKSIAYLQMNSLKTEDTAEYYCTKDSEAYYGSGSYMVGYWGQGTLVTVSS

SEQ ID NO: 26 - protein, 9 aa, Artificial Sequence
  Synthetic
FTFGDYAMS

SEQ ID NO: 27 - protein, 16 aa, Artificial Sequence
  Synthetic
WVGFITTKPYGGTTEY

SEQ ID NO: 28 - protein, 16 aa, Artificial Sequence
  Synthetic
KDSEAYYGSGSYMVGY

SEQ ID NO: 29 - protein, 110 aa, Artificial Sequence
  Synthetic
QSVLTQPPSVSLAPGKTATITCGGDNIENKDVHWYQQKPGQAPVLVIRFAADRPSGIPERFSGSSSGNTATLTISRVEAGDEADYYCQVWQSTRDHPVVVFGGGTKLTVL

SEQ ID NO: 30 - protein, 8 aa, Artificial Sequence
  Synthetic
NIENKDVH

SEQ ID NO: 31 - protein, 11 aa, Artificial Sequence
  Synthetic
LVIRFAADRPS

SEQ ID NO: 32 - protein, 12 aa, Artificial Sequence
  Synthetic
QVWQSTRDHPVV

SEQ ID NO: 33 - protein, 124 aa, Artificial Sequence
  Synthetic
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARGVYYYDSSGPRDYFDYWGQGTLVTVSS

SEQ ID NO: 34 - protein, 9 aa, Artificial Sequence
  Synthetic
GSFSGYYWS

SEQ ID NO: 35 - protein, 13 aa, Artificial Sequence
  Synthetic
WIGEINHSGSTNY

SEQ ID NO: 36 - protein, 17 aa, Artificial Sequence
  Synthetic
RGVYYYDSSGPRDYFDY

SEQ ID NO: 37 - protein, 107 aa, Artificial Sequence
  Synthetic
DIVMTQSPSSLSASVGDRVTITCQASQDISNYLNWYQQKPGKAPKLLIYDASNLKTGVPSRFSGSGSGTDFTFTITSLQPEDIGTYFCQQYGNLPYTFGQGTKLEIK

SEQ ID NO: 38 - protein, 8 aa, Artificial Sequence
  Synthetic
QDISNYLN

SEQ ID NO: 39 - protein, 11 aa, Artificial Sequence
  Synthetic
LLIYDASNLKT

SEQ ID NO: 40 - protein, 8 aa, Artificial Sequence
  Synthetic
QQYGNLPY

SEQ ID NO: 41 - protein, 119 aa, Artificial Sequence
  Synthetic
QMQLVQSGGGVVQPGRSLRLSCAASGFTFSSYGMHWVRQAPGKGLEWVAVISDDGSNKYYADSVKGRFTISRDNSKNTLYLQMNSLRAEDTAVYYCAKKDVATILFDNWGQGTLVTVSS

SEQ ID NO: 42 - protein, 9 aa, Artificial Sequence
  Synthetic
FTFSSYGMH

SEQ ID NO: 43 - protein, 14 aa, Artificial Sequence
  Synthetic
WVAVISDDGSNKYY

SEQ ID NO: 44 - protein, 11 aa, Artificial Sequence
  Synthetic
KKDVATILFDN

SEQ ID NO: 45 - protein, 107 aa, Artificial Sequence
  Synthetic
VIWMTQSPSTLSASVGDRVTITCRASQSITRWLAWYQQKPGKAPKLLIYKTSDLDGGVPSRFSGSESGAEFTLTISSLQPDDVATYFCQHYNTRSWTFGQGTKVELK

SEQ ID NO: 46 - protein, 8 aa, Artificial Sequence
  Synthetic
QSITRWLA

SEQ ID NO: 47 - protein, 11 aa, Artificial Sequence
  Synthetic
LLIYKTSDLDG

SEQ ID NO: 48 - protein, 8 aa, Artificial Sequence
  Synthetic
QHYNTRSW

SEQ ID NO: 49 - protein, 131 aa, Artificial Sequence
  Synthetic
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARGPLRKYYDFWSGSRNYYYYGMDVWGQGTTVTVSS

SEQ ID NO: 50 - protein, 9 aa, Artificial Sequence
  Synthetic
GSFSGYYWS

SEQ ID NO: 51 - protein, 13 aa, Artificial Sequence
  Synthetic
WIGEINHSGSTNY

SEQ ID NO: 52 - protein, 24 aa, Artificial Sequence
  Synthetic
RGPLRKYYDFWSGSRNYYYYGMDV

SEQ ID NO: 53 - protein, 106 aa, Artificial Sequence
  Synthetic
QPVLTQDPAVSVALGQTVRITCQGDSLRSNYASWYQLKPGQAPVLVIYDKNNRPSGIPDRFSGSNSGNTASLTIAGAQAEDEADYYCTSRDSSGTVFGGGTKLTVL

SEQ ID NO: 54 - protein, 8 aa, Artificial Sequence
  Synthetic
SLRSNYAS

SEQ ID NO: 55 - protein, 11 aa, Artificial Sequence
  Synthetic
LVIYDKNNRPS

SEQ ID NO: 56 - protein, 8 aa, Artificial Sequence
  Synthetic
TSRDSSGT

SEQ ID NO: 57 - protein, 124 aa, Artificial Sequence
  Synthetic
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARDYRGYSGQNYYGMDVWGQGTTVTVSS

SEQ ID NO: 58 - protein, 9 aa, Artificial Sequence
  Synthetic
FTFSSYSMN

SEQ ID NO: 59 - protein, 14 aa, Artificial Sequence
  Synthetic
WVSSISSSSSYIYY

SEQ ID NO: 60 - protein, 16 aa, Artificial Sequence
  Synthetic
RDYRGYSGQNYYGMDV

SEQ ID NO: 61 - protein, 110 aa, Artificial Sequence
  Synthetic
QSALTQPPSVSAAPRQKVTISCSGSSSNIGNNDVSWYQQLPGTVPKLLIYDNNKRPSGIPDRFSGSKSGTSASLGITGLQTGDEADYYCGVWDSSLSGYVFGTGTKVTVL

SEQ ID NO: 62 - protein, 10 aa, Artificial Sequence
  Synthetic
SSNIGNNDVS

SEQ ID NO: 63 - protein, 11 aa, Artificial Sequence
  Synthetic
LLIYDNNKRPS

SEQ ID NO: 64 - protein, 10 aa, Artificial Sequence
  Synthetic
GVWDSSLSGY

SEQ ID NO: 65 - protein, 121 aa, Artificial Sequence
  Synthetic
QVQLVESGGGLVKPGGSLRLSCAASGFSFNNAWMSWVRQAPGKGLEWVGRIKSKTDDATTEYAAPVEGRFTISRDDSKNMLYLQMDSLKTEDTAVYYCTTDLRWEELPMYWGQGTLVTVSS

SEQ ID NO: 66 - protein, 9 aa, Artificial Sequence
  Synthetic
FSFNNAWMS

SEQ ID NO: 67 - protein, 16 aa, Artificial Sequence
  Synthetic
WVGRIKSKTDDATTEY

SEQ ID NO: 68 - protein, 11 aa, Artificial Sequence
  Synthetic
TDLRWEELPMY

SEQ ID NO: 69 - protein, 108 aa, Artificial Sequence
  Synthetic
VIWMTQSPSSLSASVGDRVTITCRASQGIRNDLGWYQQKPGKAPKVLIYAASSLQSGVPSRFSGSGSGTDFTLTISSLQPEDFATYYCLQDYNYPQTFGQGTKVGIKR

SEQ ID NO: 70 - protein, 8 aa, Artificial Sequence
  Synthetic
QGIRNDLG

SEQ ID NO: 71 - protein, 11 aa, Artificial Sequence
  Synthetic
VLIYAASSLQS

SEQ ID NO: 72 - protein, 8 aa, Artificial Sequence
  Synthetic
LQDYNYPQ

SEQ ID NO: 73 - protein, 124 aa, Artificial Sequence
  Synthetic
EVQLVESGGGLVEPGGSLRLSCAASGFTFSSYSMSWVRQAPGKGLEWVGRIKSETDGGTTDYAGPAKGKFIISRNDAENTVSLQMNSLKFEDTAVYHCTTDSSSSWFSYSFDNWGQETLVTVSS

SEQ ID NO: 74 - protein, 9 aa, Artificial Sequence
  Synthetic
FTFSSYSMS

SEQ ID NO: 75 - protein, 16 aa, Artificial Sequence
  Synthetic
WVGRIKSETDGGTTDY

SEQ ID NO: 76 - protein, 14 aa, Artificial Sequence
  Synthetic
TDSSSSWFSYSFDN

SEQ ID NO: 77 - protein, 107 aa, Artificial Sequence
  Synthetic
EIVMTQSPATLSVSPGERATLSCRASQNVNSNLAWYQQKTGQAPRLLIFGASTRATGIPARFSGSGSGTEFTLTIGSLQSEDFAVYYCQQYNNWPPTFGGWTKVEIK

SEQ ID NO: 78 - protein, 8 aa, Artificial Sequence
  Synthetic
QNVNSNLA

SEQ ID NO: 79 - protein, 11 aa, Artificial Sequence
  Synthetic
LLIFGASTRAT

SEQ ID NO: 80 - protein, 8 aa, Artificial Sequence
  Synthetic
QQYNNWPP

SEQ ID NO: 81 - protein, 124 aa, Artificial Sequence
  Synthetic
QLQLQESGPGLVKPAETLSLTCTVSGGSVSSGDNLWDWIRQPPGKGLEWIGSIRDSGNPAYNPSLRSRVTISADTSTDQFSLKLTSVTAADTAVYYCARHSDGGYGKIYGMDVWGQGTTVTVSS

SEQ ID NO: 82 - protein, 11 aa, Artificial Sequence
  Synthetic
GSVSSGDNLWD

SEQ ID NO: 83 - protein, 13 aa, Artificial Sequence
  Synthetic
WIGSIRDSGNPAY

SEQ ID NO: 84 - protein, 15 aa, Artificial Sequence
  Synthetic
RHSDGGYGKIYGMDV

SEQ ID NO: 85 - protein, 107 aa, Artificial Sequence
  Synthetic
DIQLTQSPSFLSASVGDRVTITCRASQAVSTYLVWYLQKPGQAPRLLIYAASTLQSGVPSRFSGSGSGTDFTLTISRLQPEDAGTYFCQQLNSNALVFGGGTKVDIK

SEQ ID NO: 86 - protein, 8 aa, Artificial Sequence
  Synthetic
QAVSTYLV

SEQ ID NO: 87 - protein, 11 aa, Artificial Sequence
  Synthetic
LLIYAASTLQS

SEQ ID NO: 88 - protein, 9 aa, Artificial Sequence
  Synthetic
QQLNSNALV

SEQ ID NO: 89 - protein, 120 aa, Artificial Sequence
  Synthetic
QVQLVQSGAEVKKPGASVKVSCKASGYTFTDYYMHWVRQAPGQGLEWMGWINPNSGGTNYAQKFQGRVTMTRDTSITTAYMELSWLRSDDTAVYYCAREVGTTTTGVDYWGQGTLVTVSS

SEQ ID NO: 90 - protein, 9 aa, Artificial Sequence
  Synthetic
YTFTDYYMH

SEQ ID NO: 91 - protein, 15 aa, Artificial Sequence
  Synthetic
WMGWINPNSGGTNYA

SEQ ID NO: 92 - protein, 12 aa, Artificial Sequence
  Synthetic
REVGTTTTGVDY

SEQ ID NO: 93 - protein, 107 aa, Artificial Sequence
  Synthetic
VIWMTQSPSSLSASVGDRVTFTCQASQDINIYLTWYQIKPGKAPKVLIYDASTLESGVPSRFSGSGFGTHFVFTISSLQPEDFATYYCQHYDSLPLTFGGGTKVEIK

SEQ ID NO: 94 - protein, 8 aa, Artificial Sequence
  Synthetic
QDINIYLT

SEQ ID NO: 95 - protein, 11 aa, Artificial Sequence
  Synthetic
VLIYDASTLES

SEQ ID NO: 96 - protein, 8 aa, Artificial Sequence
  Synthetic
QHYDSLPL

(19) United States
(12) Patent Application Publication
(10) Pub. No.: US 2024/0336683 A1
(43) Pub. Date: Oct. 10, 2024

(54) ANTIBODIES SPECIFIC TO SIALIC ACID-BINDING IG-LIKE LECTIN 15 AND USES THEREOF

(71) Applicant: Elpis Biopharmaceuticals, Lexington, MA (US)

(72) Inventors: Kehao ZHAO, Lexington, MA (US); Yan CHEN, Lexington, MA (US); Samuel Clement HASSAN, Lexington, MA (US); Jenna NGUYEN, Lexington, MA (US); Ning JIANG, Lexington, MA (US)

(73) Assignee: Elpis Biopharmaceuticals, Lexington, MA (US)

(21) Appl. No.: 18/551,224

(22) PCT Filed: Mar. 18, 2022
(86) PCT No.: PCT/US2022/020937

§ 371 Date: Sep. 19, 2023

Related U.S. Application Data
(60) Provisional application No. 63/163,680, filed on Mar. 19, 2021.

(51) Int. Cl. C07K 16/28; A61K 39/00; A61P 35/00; G01N 33/574; G01N 33/58; G01N 33/68

(52) U.S. Cl. CPC C07K 16/2803; A61P 35/00; G01N 33/574; G01N 33/58; G01N 33/6893; A61K 2039/505; C07K 2317/21; C07K 2317/24; C07K 2317/565; C07K 2317/622; C07K 2317/76; C07K 2317/92

(57) ABSTRACT

Disclosed herein are high affinity anti-Siglec 15 antibodies and methods of using such for therapeutic and/or diagnostic purposes. Also provided herein are methods for producing such anti-Siglec 15 antibodies.

### CROSS-REFERENCE TO RELATED APPLICATIONS

This application claims the benefit of priority to U.S. Provisional Patent Application No. 63/163,680, filed Mar. 19, 2021, which is hereby incorporated by reference in its entirety.

### BACKGROUND OF THE INVENTION

Sialic acid-binding Ig-like lectin 15 (Siglec15) is a member of the Siglec family of glycan-recognition proteins. Siglec proteins are expressed on various types of leukocytes and play important roles in modulating immune responses via binding to ligands at the extracellular domain and mediating intracellular signaling transduction.

Siglec15 was found to be upregulated on human cancer cells and tumor-infiltrating myeloid cells. It has been reported that Siglec15 is an immune suppressor that suppresses antigen-specific T cell responses. Accordingly, Siglec 15 is suggested to be a potential therapeutic target for cancer immunotherapy.

### SUMMARY OF THE INVENTION

The present disclosure is based, at least in part, on the development of antibodies binding to human sialic acid-binding Ig-like lectin 15 (Siglec15). Such anti-Siglec15 antibodies showed high binding affinity and specificity to human Siglec15. Further, certain exemplary antibodies (e.g., clone 2020EP32-H11) are capable of inducing antibody-dependent cytotoxicity (ADCC) against cells expressing Siglec 15 and activating immune cells. Further, the exemplary H11 clone (in monoclonal antibody format) showed desirable pharmacokinetic features (e.g., long half-life and little toxicity) as observed in cynomolgus monkeys. Accordingly, the anti-Siglec15 antibodies disclosed herein would be expected to have high therapeutic effects via modulating immune responses and/or neutralizing Siglec 15-positive cells or Siglec15 dependent signals.

Accordingly, one aspect of the present disclosure features an isolated antibody that binds sialic acid-binding Ig-like lectin 15 (Siglec15). Such an antibody binds to the same epitope as a reference antibody or competes against the reference antibody from binding to Siglec15. The reference antibody can be one of the following: 2019EP47-A02, 2019EP47-A05, 2019EP47-A10, 2019EP47-C12, 2020EP032-A08, 2020EP032-A12, 2020EP032-B03, 2020EP032-H11, 2020EP032-C09, 2020EP083-G11, 2020EP083-H01, and 2020EP085-G5.

In some embodiments, the anti-Siglec15 antibody disclosed herein may comprise: (a) a heavy chain complementary determining region 1 (HC CDR1), a heavy chain complementary determining region 2 (HC CDR2), and a heavy chain complementary determining region 3 (HC CDR3), wherein the HC CDR1, HC CDR2, and HC CDR3 collectively are at least 80% identical to the heavy chain CDRs of the reference antibody; and/or (b) a light chain complementary determining region 1 (LC CDR1), a light chain complementary determining region 2 (LC CDR2), and a light chain complementary determining region 3 (LC CDR3), wherein the LC CDR1, LC CDR2, and LC CDR3 collectively are at least 80% identical to the light chain CDRs of the reference antibody. In some examples, the HC CDRs of the anti-Siglec15 antibody disclosed herein collectively contain no more than 8 amino acid residue variations as compared with the HC CDRs of the reference antibody. Alternatively or in addition, the LC CDRs of the anti-Siglec15 antibody collectively contain no more than 8 amino acid residue variations as compared with the LC CDRs of the reference antibody.

In some instances, the anti-Siglec15 antibody disclosed herein may comprise a V_{H} that is at least 85% identical to the V_{H} of the reference antibody, and/or a V_{L} that is at least 85% identical to the V_{L} of the reference antibody. In some instances, the anti-Siglec 15 antibody has a binding affinity of less than about 50 nM to Siglec-15 expressed on cell surface. In some examples, the anti-Siglec15 antibody has a binding affinity less than 10 nM. In some examples, the anti-Siglec15 antibody has a binding affinity less than 5 nM to Siglec15 expressed on cell surface. In some examples, the anti-Siglec 15 antibody has a binding affinity less than 1.5 nM to the Siglec15 expressed on cell surface.

In some examples, the anti-Siglec 15 antibody disclosed herein comprises the same heavy chain complementary determining regions (HC CDRs) and the same light chain complementary determining regions (LC CDRs) as the reference antibody. In specific examples, the anti-Siglec 15 antibody comprises the same V_{H} and the same V_{L} as the reference antibody.

Any of the anti-Siglec 15 antibodies disclosed herein may be a human antibody. Alternatively, it may be a humanized antibody. In some instances, the anti-Siglec 15 antibody is a full-length antibody. Alternatively, it may be an antigen-binding fragment thereof. In some instances, the anti-Siglec15 antibody may be a single-chain variable fragment (scFv) antibody. In some instances, the antibody is a fusion polypeptide comprising the scFv.

In another aspect, the present disclosure features a nucleic acid or a set of nucleic acids, which collectively encodes any of the anti-Siglec15 antibodies disclosed herein. In some instances, a set of nucleic acids disclosed herein refers to two nucleic acid molecules each encoding one chain of the antibody and collectively encoding the heavy and light chain of the antibody. In some embodiments, the nucleic acid or the set of nucleic acids is a vector or a set of vectors, for example, an expression vector or an expression vector set. Also provided herein is a host cell comprising any of the nucleic acids or the set of nucleic acids coding for any of the anti-Siglec 15 antibodies disclosed herein.

In yet another aspect, the present disclosure features a pharmaceutical composition comprising any of the anti-Siglec15 antibodies disclosed herein, any of the coding nucleic acids, of any one of claims 1-12, the nucleic acid or nucleic acids of any one of claims 13-15, or the host cell of claim 16, and a pharmaceutically acceptable carrier.

In addition, the present disclosure provides a method for inhibiting Siglec-15 or Siglec-15⁺ cells in a subject, comprising administering to a subject in need thereof any effective amount of any of the pharmaceutical compositions disclosed herein. In some embodiments, the pharmaceutical composition comprises an anti-Siglec15 antibody as disclosed herein. In some embodiments, the subject is a human patient having Siglec15+ disease cells, e.g., tumor cells or immune cells. In some examples, the human patient has a Siglec15+ cancer. Examples include non-small cell lung cancer (NSCLC), ovarian cancer, breast cancer, head-and-neck cancer, renal carcinoma, pancreatic cancer, endometrial cancer, urothelial cancer, thyroid cancer, colon cancer, colorectal cancer, melanoma, liver cancer, or gastric cancer.

Moreover, the present disclosure provides a method for detecting presence of Siglec-15, comprising: (i) contacting an anti-Siglec15 antibody as disclosed herein with a sample suspected of containing Siglec-15, and (ii) detecting binding of the antibody to Siglec-15. In some instances, the anti-Siglec15 antibody may be conjugated to a detectable label. In some instances, the Siglec-15 is expressed on cell surface. In some embodiments, the contacting step is performed by administering the antibody to a subject.

The present disclosure also features a method of producing an antibody binding to Siglec-15, comprising: (i) culturing a host cell comprising nucleic acid(s) encoding any of the anti-Siglec 15 antibodies disclosed herein under conditions allowing for expression of the antibody that binds Siglec-15; and (ii) harvesting the antibody thus produced from the cell culture.

Also within the present disclosure are anti-Siglec15 antibodies or pharmaceutical compositions comprising such for use in treating a disease or disorder associated with Siglec15, for example, cancer or an immune disorder, or use of such an antibody for manufacturing a medicament for use in treating the target disease.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIG. 1 is a diagram showing anti-Siglec15 IgG antibodies binding to Siglec15/HEK293 by FACS analysis.

FIGS. 2A-2D include diagrams showing epitope binning between 2020EP32-H11 and 5G12 antibodies. FIG. 2A: competitive binding of anti-Siglec15 2020EP32-H11 IgG antibody to Siglec 15 in the presence of 5G12. FIG. 2B: competitive binding of anti-Siglec15 5G12 antibody to Siglec15 in the presence of 2020EP32-H11. FIG. 2C: competitive binding of anti-Siglec 15 2019EP47-A02 IgG antibody to Siglec 15 in the presence of 5G12. FIG. 2D: competitive binding of anti-Siglec15 2019EP47-A02 IgG antibody to Siglec 15 in the presence of 2020EP32-H11.

FIGS. 3A-C include diagrams showing binding specificity of anti-Siglec15 antibody 2020EP32-H11. FIG. 3A: a diagram showing binding activity of 2020EP32-H11 to various Siglec proteins as determined by surface plasmon resonance (SPR). FIG. 3B: a diagram showing binding activity of 2020EP32-H11 to various siglec proteins as measured by ELISA. FIG. 3C: a diagram showing binding activity of antibody 5G12 to various siglec proteins as determined by ELISA.

FIGS. 4A-4D include diagrams showing the activity of exemplary anti-Siglec 15 antibodies (IgG) in activating T cells. FIGS. 4A-4B: activity of exemplary anti-Siglec 15 IgG antibodies as indicated in inducing proliferation of T-Cells in human PBMCs. FIGS. 4C-4D: T-cell activation activity of exemplary antibody 2020EP32-H11 as compared with reference antibody 5G12, using human PBMCs.

FIGS. 5A-5D include diagrams comparing T cell activation activity of antibody 2020EP32-H11 with that of antibody 5G12 using human PBMCs from donor #559. FIG. 5A: CD3+ cell proliferation. FIG. 5B: CD4+ cell proliferation. FIG. 5C: Treg cell proliferation. FIG. 5D: CD8+ cell proliferation.

FIGS. 6A-6G include diagrams showing that exemplary anti-Siglec 15 IgG antibodies activate NK cells in a dose-dependent manner. FIG. 6A: NK cell proliferation activity of antibody 2020EP32-H11 as compared with antibody 5G12, using PBMCs from donor #559. FIG. 6B: Interferon gamma (IFNγ) secretion induced by antibody H11 as compared with antibody 5G12, by PBMCs from donor #559. FIG. 6C: TNF-α secretion induced by antibody 2020EP32-H11 as compared with antibody 5G12, by PBMCs from donor #559. FIG. 6D: NK proliferation activity of antibody 2020EP32-H11 as compared with antibody 5G12 using PBMCs from Donor 211. FIG. 6E: NK proliferation activity of antibody 2020EP32-H11 as compared with antibody 5G12 using PBMCs from Donor 938. FIG. 6F: TNF-α secretion induced by antibody 2020EP32-H11 as compared with antibody 5G12, by PBMCs from donor #211. FIG. 6G: TNF-α secretion induced by antibody 2020EP32-H11 as compared with antibody 5G12, by PBMCs from donor #938.

FIGS. 7A-7M include diagrams showing ADCC activity of exemplary anti-Siglec15 IgG antibodies in a dose-dependent manner. FIG. 7A: ADCC effects against MC38-hSiglec15 cell line using Jurkat NFAT Luciferase assay in the presence of anti-Siglec 15 antibodies as indicated. FIG. 7B: ADCC effects against B16F10-hSiglec15 cell line by NK cells from Donor #066. FIG. 7C: INFγ section by NKs from Donor #066 when incubated with B16F10 or B16F10-hSiglec15 cells in the presence of antibody 2020EP32-H11 or 5G12. FIG. 7D: TNF-α section by NK cells from Donor #066 when incubated with B16F10 or B16F10-hSiglec15 cells in the presence of antibody 2020EP32-H11 or 5G12. FIG. 7E: ADCC effects against B16F10-hSiglec 15 cell line by NK cells from Donor #993. FIG. 7F: INFγ section by NKs from Donor #993 when incubated with B16F10 or B16F10-hSiglec15 cells in the presence of antibody 2020EP32-H11 or 5G12. FIG. 7G: TNF-α section by NK cells from Donor #993 when incubated with B16F10 or B16F10-hSiglec15 cells in the presence of antibody 2020EP32-H11 or 5G12. FIG. 7H: ADCC effects against MC38-hSiglec 15 cell line by NK cells from Donor #033. FIG. 7I: INFγ section by NKs from Donor #033 when incubated with B16F10 or B16F10-hSiglec15 cells in the presence of antibody 2020EP32-H11 or 5G12. FIG. 7J: TNF-α section by NK cells from Donor #033 when incubated with B16F10 or B16F10-hSiglec15 cells in the presence of antibody 2020EP32-H11 or 5G12. FIG. 7K: ADCC effects against MC38-hSiglec15 cell line by NK cells from Donor #054. FIG. 7L: INFγ section by NKs from Donor #054 when incubated with B16F10 or B16F10-hSiglec15 cells in the presence of antibody 2020EP32-H11 or 5G12. FIG. 7M: TNF-α section by NK cells from Donor #054 when incubated with B16F10 or B16F10-hSiglec15 cells in the presence of antibody 2020EP32-H11 or 5G12.

FIGS. 8A-8D include diagrams showing immune cell profiling in B16F10-hSiglec15 tumor bearing mice treated with the 2020EP32-H11 antibody or 5G12 antibody. FIG. 8A: NK cells. FIG. 8B: M2 macrophage. FIG. 8C: CD8⁺ T cells. FIG. 8D: T_{reg} cells.

FIGS. 9A-9B include diagrams illustrating pharmacokinetic (PK) analysis of exemplary antibody H11 mAb in Cynomolgus monkey. FIG. 9A: concentration of H11 in plasma over time. FIG. 9B: body weight change of the money over time.

### DETAILED DESCRIPTION OF THE INVENTION

Provided herein are antibodies capable of binding to human Siglec 15 (“anti-Siglec15 antibodies”), particularly Siglec 15 expressed on cell surface. The anti-Siglec15 antibodies disclosed herein show high binding affinity and specificity to human Siglec15 (e.g., cell-surface Siglec15), high bioactivity in, for example, ADCC effects and immune activating effects, both in vitro and in vivo. Further, when compared with a control anti-Siglec 15 antibody 5G12, certain exemplary anti-Siglec 15 antibodies disclosed herein (e.g., clone 2020EP32-H11) showed better binding activity and bioactivities. The anti-Siglec 15 antibodies disclosed herein are expected to show superior anti-cancer and/or immune modulating effects.

Siglec 15 is a member of the Siglec family, which primarily is expressed on various myeloid cells. Siglec-15 has an extracellular domain consisting of two immunoglobulin-like domains, followed by a transmembrane domain that contains a lysine residue (Lys274 in human Siglec15) that is essential for the interaction with adapter protein DAP12, and a cytoplasmic tail. Siglec 15 proteins from various species are well known in the art. For example, the amino acid sequence of human Siglec15 can be found in GenBank under Gene ID: 284266.

Siglec 15 is found to be a critical immune suppressor and is upregulated on various types of cancer. It thus becomes a potential target for cancer immunotherapy and/or modulation of immune responses.

### I. Antibodies Binding to Siglec15

The present disclosure provides antibodies binding to Siglec 15, for example, human Siglec15. In some embodiments, the anti-Siglec15 antibodies disclosed herein are capable of binding to Siglec 15 expressed on cell surface. As such, the antibodies disclosed herein may be used for either therapeutic or diagnostic purposes to target Siglec15-positive cells (e.g., cancer cells or immune cells). As used herein, the term “anti-Siglec15 antibody” refers to any antibody capable of binding to a Siglec15 polypeptide (e.g., a Siglec15 polypeptide expressed on cell surface), which can be of a suitable source, for example, human or a non-human mammal (e.g., mouse, rat, rabbit, primate such as monkey, etc.).

An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody”, e.g., anti-Siglec15 antibody, encompasses not only intact (e.g., full-length) polyclonal or monoclonal antibodies, but also antigen-binding fragments thereof (such as Fab, Fab′, F(ab′)2, Fv), single-chain antibody (scFv), fusion proteins comprising an antibody portion, humanized antibodies, chimeric antibodies, diabodies, single domain antibody (e.g., nanobody), single domain antibodies (e.g., a V_{H} only antibody), multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody, e.g., anti-Siglec15 antibody, includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

A typical antibody molecule comprises a heavy chain variable region (V_{H}) and a light chain variable region (V_{L}), which are usually involved in antigen binding. The V_{H} and V_{L} regions can be further subdivided into regions of hypervariability, also known as “complementarity determining regions” (“CDR”), interspersed with regions that are more conserved, which are known as “framework regions” (“FR”). Each V_{H} and V_{L} is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also hgmp.mrc.ac.uk and bioinf.org.uk/abs).

The anti-Siglec antibody described herein may be a full-length antibody, which contains two heavy chains and two light chains, each including a variable domain and a constant domain. Alternatively, the anti-Siglec15 antibody can be an antigen-binding fragment of a full-length antibody. Examples of binding fragments encompassed within the term “antigen-binding fragment” of a full length antibody include (i) a Fab fragment, a monovalent fragment consisting of the V_{L}, V_{H}, C_{L} and C_{H}1 domains; (ii) a F(ab′)₂ fragment, a bivalent fragment including two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR) that retains functionality. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules known as single chain Fv (scFv). See e.g., Bird et al. (1988) Science 242:423-426; and Huston et al. (1988) Proc. Natl. Acad. Sci. USA 85:5879-5883.

The antibodies described herein can be of a suitable origin, for example, murine, rat, or human. Such antibodies are non-naturally occurring, i.e., would not be produced in an animal without human act (e.g., immunizing such an animal with a desired antigen or fragment thereof or isolated from antibody libraries). Any of the antibodies described herein, e.g., anti-Siglec15 antibody, can be either monoclonal or polyclonal. A “monoclonal antibody” refers to a homogenous antibody population and a “polyclonal antibody” refers to a heterogeneous antibody population. These two terms do not limit the source of an antibody or the manner in which it is made.

In some embodiments, the anti-Siglec15 antibodies are human antibodies, which may be isolated from a human antibody library or generated in transgenic mice. For example, fully human antibodies can be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are Xenomouse™ from Amgen, Inc. (Fremont, Calif.) and HuMAb-Mouse™ and TC Mouse™ from Medarex, Inc. (Princeton, N.J.). In another alternative, antibodies may be made recombinantly by phage display or yeast technology. See, for example, U.S. Pat. Nos. 5,565,332; 5,580,717; 5,733,743; and 6,265,150; and Winter et al., (1994) Annu. Rev. Immunol. 12:433-455. Alternatively, the antibody library display technology, such as phage, yeast display, mammalian cell display, or mRNA display technology as known in the art can be used to produce human antibodies and antibody fragments in vitro, from immunoglobulin variable (V) domain gene repertoires from unimmunized donors.

In other embodiments, the anti-Siglec15 antibodies may be humanized antibodies or chimeric antibodies. Humanized antibodies refer to forms of non-human (e.g., murine) antibodies that are specific chimeric immunoglobulins, immunoglobulin chains, or antigen-binding fragments thereof that contain minimal sequence derived from non-human immunoglobulin. In general, humanized antibodies are human immunoglobulins (recipient antibody) in which residues from a CDR of the recipient are replaced by residues from a CDR of a non-human species (donor antibody) such as mouse, rat, or rabbit having the desired specificity, affinity, and capacity. In some instances, one or more Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In some instances, the humanized antibody may comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, or six) which are altered with respect to the original antibody, which are also termed one or more CDRs “derived from” one or more CDRs from the original antibody. Humanized antibodies may also involve affinity maturation. Methods for constructing humanized antibodies are also well known in the art. See, e.g., Queen et al., Proc. Natl. Acad. Sci. USA, 86:10029-10033 (1989).

In some embodiments, the anti-Siglec15 antibody disclosed herein can be a chimeric antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region. Techniques developed for the production of “chimeric antibodies” are well known in the art. See, e.g., Morrison et al. (1984) Proc. Natl. Acad. Sci. USA 81, 6851; Neuberger et al. (1984) Nature 312, 604; and Takeda et al. (1984) Nature 314:452.

In some embodiments, the anti-Siglec15 antibodies described herein specifically bind to the corresponding target antigen (e.g., human Siglec15) or an epitope thereof. An antibody that “specifically binds” to an antigen or an epitope is a term well understood in the art. A molecule is said to exhibit “specific binding” if it reacts more frequently, more rapidly, with greater duration and/or with greater affinity with a particular target antigen than it does with alternative targets. An antibody “specifically binds” to a target antigen or epitope if it binds with greater affinity, avidity, more readily, and/or with greater duration than it binds to other substances. For example, an antibody that specifically (or preferentially) binds to an antigen (Siglec 15 such as human Siglec15) or an antigenic epitope therein is an antibody that binds this target antigen with greater affinity, avidity, more readily, and/or with greater duration than it binds to other antigens or other epitopes in the same antigen. It is also understood with this definition that, for example, an antibody that specifically binds to a first target antigen may or may not specifically or preferentially bind to a second target antigen. As such, “specific binding” or “preferential binding” does not necessarily require (although it can include) exclusive binding. In some examples, an antibody that “specifically binds” to a target antigen or an epitope thereof may not bind to other antigens or other epitopes in the same antigen (i.e., only baseline binding activity can be detected in a conventional method).

In some embodiments, an anti-Siglec 15 antibody as described herein has a suitable binding affinity for the target antigen (e.g., human Siglec15) or antigenic epitopes thereof. As used herein, “binding affinity” refers to the apparent association constant or K_{A}. The K_{A} is the reciprocal of the dissociation constant (K_{D}). The anti-Siglec15 antibody described herein may have a binding affinity (K_{D}) of at least 100 nM, 50 nM, 10 nM, 1 nM, 0.1 nM, or lower for Siglec15. In some instances, the anti-Siglec 15 antibody disclosed herein may have a binding affinity less than 10 nM, less than 5 nM, less than 2 nM, or less than 1 nm for cell surface Siglec15. An increased binding affinity corresponds to a decreased K_{D}. Higher affinity binding of an antibody for a first antigen relative to a second antigen can be indicated by a higher K_{A} (or a smaller numerical value K_{D}) for binding the first antigen than the K_{A} (or numerical value K_{D}) for binding the second antigen. In such cases, the antibody has specificity for the first antigen (e.g., a first protein in a first conformation or mimic thereof) relative to the second antigen (e.g., the same first protein in a second conformation or mimic thereof; or a second protein). Differences in binding affinity (e.g., for specificity or other comparisons) can be at least 1.5, 2, 3, 4, 5, 10, 15, 20, 37.5, 50, 70, 80, 90, 100, 500, 1000, 10,000 or 10⁵ fold. In some embodiments, any of the anti-Siglec 15 antibodies may be further affinity matured to increase the binding affinity of the antibody to the target antigen or antigenic epitope thereof.

Binding affinity (or binding specificity) can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in HBS-P buffer (10 mM HEPES pH7.4, 150 mM NaCl, 0.005% (v/v) Surfactant P20). These techniques can be used to measure the concentration of bound binding protein as a function of target protein concentration. The concentration of bound binding protein ([Bound]) is generally related to the concentration of free target protein ([Free]) by the following equation:

$$[ Bound ] = [ Free ] / ( K d + [ Free ] )$$

It is not always necessary to make an exact determination of K_{A}, though, since sometimes it is sufficient to obtain a quantitative measurement of affinity, e.g., determined using a method such as ELISA or FACS analysis, is proportional to K_{A}, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

In some embodiments, the anti-Siglec15 antibody disclosed herein has an EC₅₀ value of lower than 10 nM, e.g., <2 nM, <1 nM, <0.5 nM, or lower than 0.1 nM, for binding to Siglec15-positive cells. As used herein, EC₅₀ values refer to the minimum concentration of an antibody required to bind to 50% of the cells in a Siglec 15-positive cell population. EC₅₀ values can be determined using conventional assays and/or assays disclosed herein. See, e.g., Examples below.

A number of exemplary anti-Siglec15 antibodies are described in the present disclosure and provided by amino acid sequence as below, namely antibodies: 2019EP47-A02, 2019EP47-A05, 2019EP47-A10, 2019EP47-C12, 2020EP032-A08, 2020EP032-A12, 2020EP032-B03, 2020EP032-H11, 2020EP032-C09, 2020EP083-G11, 2020EP083-H01, and 2020EP085-G5.

Table 1 below lists the amino acid sequences of the heavy chain variable region and light chain variable region of the exemplary anti-Siglec15 antibodies. Their heavy chain and light chain complementary determining regions (CDRs) within the V_{H} and V_{L} domains are also identified (determined by the Kabat scheme). See also www2.mrc-lmb.cam.ac.uk/vbase/alignments2.php.

TABLE 1
Structural Information of Exemplary Anti-Siglec15 Antibodies

| Antibody | | | SEQ ID |
|---|---|---|---|
| Clones | | Amino Acid Sequence | NO: |
| 2019EP47- | VH | EVQLVQSGGGVVQPGRSLRLSCAASGFTFSSYAMHWVRQAPGKGLEWVAVISY | 1 |
| A02- | | DGSNKYYADSVKGRFTISRDNSKNTLYLQMNSLRAEDTAVYYCARSRYSSSLR | |
| | | RYYGMDVWGQGTTVTVSS | |
| | HCDR1 | FTFSSYAMH | 2 |
| | HCDR2 | WVAVISYDGSNKYY | 3 |
| | HCDR3 | RSRYSSSLRRYYGMDV | 4 |
| | VL | AIQLTQSPSSLSASVGDRVTITCQATHDIANYLNWYQQKPGKAPKLLIYAASN | 5 |
| | | LETGVPSRFSGSGSGTDFTFAISSLQPEDIAVYYCQQYDNLPLTFGGGTKVEI | |
| | | K | |
| | LCDR1 | HDIANYLN | 6 |
| | LCDR2 | LLIYAASNLET | 7 |
| | LCDR3 | QQYDNLPL | 8 |
| 2019EP47- | VH | QMQLVQSGAEVKKPGSSVKVSCKASGGTFSSYAISWVRQAPGQGLEWMGRIIP | 9 |
| A05 | | ILGIANYAQKFQGRVTITADKSTSTAYMELSSLRSEDTAVYYCAVNTYYYDSS | |
| | | GYYYEYFDYWGQGTLVTVSS | |
| | HCDR1 | GTFSSYAIS | 10 |
| | HCDR2 | WMGRIIPILGIANY | 11 |
| | HCDR3 | VNTYYYDSSGYYYEYFDY | 12 |
| | VL | EIVLTQSPSSLSASVGDRVTITCQASQHINNHLNWYQQKPGKGPKLLIYDVSN | 13 |
| | | LEAGVPSRFSASGSGTDFSFTISSLQPEDFATYYCQQYDSLPLTFGQGTKVEV | |
| | | K | |
| | LCDR1 | QHINNHLN | 14 |
| | LCDR2 | LLIYDVSNLEA | 15 |
| | LCDR3 | QQYDSLPL | 16 |
| 2019EP47- | VH | QMQLVQSGGGVVQPGRSLRLSCAASGFTFSSYGMHWVRQAPGKGLEWVAVISY | 17 |
| A10 | | DGSNKYYADSVKDRFTISRDNSKNTLYLQMNSLRAEDTAVYYCAKDIVGDSGS | |
| | | YPIYYYYGMDVWGQGTTVTVSS | |
| | HCDR1 | FTFSSYGMH | 18 |
| | HCDR2 | WVAVISYDGSNKYY | 19 |
| | HCDR3 | KDIVGDSGSYPIYYYYGMDV | 20 |
| | VL | SQSALTQPHSVSESPGKTVTISCTRSSGSIASNYVQWYQQRPGSAPSTVIYED | 21 |
| | | NQRPSGVPDRFSGSVDISSNSASLTISGLQTEDEADYYCQSYDNDNMVEGGGT | |
| | | KLTVL | |
| | LCDR1 | SGSIASNYVQ | 22 |
| | LCDR2 | STVIYEDNQRPS | 23 |
| | LCDR3 | QSYDNDNM | 24 |
| 2019EP47- | VH | EVQLVESGGGLEKPGRSLRLSCTASGFTFGDYAMSWFRQAPGKGLEWVGFITT | 25 |
| C12 | | KPYGGTTEYAASVKGRFTISRDDSKSIAYLQMNSLKTEDTAEYYCTKDSEAYY | |
| | | GSGSYMVGYWGQGTLVTVSS | |
| | HCDR1 | FTFGDYAMS | 26 |
| | HCDR2 | WVGFITTKPYGGTTEY | 27 |
| | HCDR3 | KDSEAYYGSGSYMVGY | 28 |
| | VL | QSVLTQPPSVSLAPGKTATITCGGDNIENKDVHWYQQKPGQAPVLVIRFAADR | 29 |
| | | PSGIPERFSGSSSGNTATLTISRVEAGDEADYYCQVWQSTRDHPVVVFGGGTK | |
| | | LTVL | |
| | LCDR1 | NIENKDVH | 30 |
| | LCDR2 | LVIRFAADRPS | 31 |
| | LCDR3 | QVWQSTRDHPVV | 32 |
| 2020EP032- | VH | QVQLQQWGAGLLKPSETLSLTCAVYGGSFSGYYWSWIRQPPGKGLEWIGEINH | 33 |
| A08 | | SGSTNYNPSLKSRVTISVDTSKNQFSLKLSSVTAADTAVYYCARGVYYYDSSG | |
| | | PRDYFDYWGQGTLVTVSS | |
| | HCDR1 | GSFSGYYWS | 34 |
| | HCDR2 | WIGEINHSGSTNY | 35 |
| | HCDR3 | RGVYYYDSSGPRDYFDY | 36 |
| | VL | DIVMTQSPSSLSASVGDRVTITCQASQDISNYLNWYQQKPGKAPKLLIYDASN | 37 |
| | | LKTGVPSRFSGSGSGTDFTFTITSLQPEDIGTYFCQQYGNLPYTFGQGTKLEI | |
| | | K | |
| | LCDR1 | QDISNYLN | 38 |
| | LCDR2 | LLIYDASNLKT | 39 |
| | LCDR3 | QQYGNLPY | 40 |
| 2020EP032- | VH | QMQLVQSGGGVVQPGRSLRLSCAASGFTFSSYGMHWVRQAPGKGLEWVAVISD | 41 |
| A12 | | DGSNKYYADSVKGRFTISRDNSKNTLYLQMNSLRAEDTAVYYCAKKDVATILF | |
| | | DNWGQGTLVTVSS | |
| | HCDR1 | FTFSSYGMH | 42 |
| | HCDR2 | WVAVISDDGSNKYY | 43 |
| | HCDR3 | KKDVATILFDN | 44 |
| | VL | VIWMTQSPSTLSASVGDRVTITCRASQSITRWLAWYQQKPGKAPKLLIYKTSD | 45 |
| | | LDGGVPSRFSGSESGAEFTLTISSLQPDDVATYFCQHYNTRSWTFGQGTKVEL | |
| | | K | |
| | LCDR1 | QSITRWLA | 46 |
| | LCDR2 | LLIYKTSDLDG | 47 |
| | LCDR3 | QHYNTRSW | 48 |
| 2020EP032- | VH | QVQLQQWGAGLLKPSETLSLTCAVYGGSFSGYYWSWIRQPPGKGLEWIGEINH | 49 |
| B03 | | SGSTNYNPSLKSRVTISVDTSKNQFSLKLSSVTAADTAVYYCARGPLRKYYDF | |
| | | WSGSRNYYYYGMDVWGQGTTVTVSS | |
| | HCDR1 | GSFSGYYWS | 50 |
| | HCDR2 | WIGEINHSGSTNY | 51 |
| | HCDR3 | RGPLRKYYDFWSGSRNYYYYGMDV | 52 |
| | VL | QPVLTQDPAVSVALGQTVRITCQGDSLRSNYASWYQLKPGQAPVLVIYDKNNR | 53 |
| | | PSGIPDRFSGSNSGNTASLTIAGAQAEDEADYYCTSRDSSGTVFGGGTKLTVL | |
| | LCDR1 | SLRSNYAS | 54 |
| | LCDR2 | LVIYDKNNRPS | 55 |
| | LCDR3 | TSRDSSGT | 56 |
| 2020EP032- | VH | EVQLVESGGGLVKPGGSLRLSCAASGFTFSSYSMNWVRQAPGKGLEWVSSISS | 57 |
| H11 | | SSSYIYYADSVKGRFTISRDNAKNSLYLQMNSLRAEDTAVYYCARDYRGYSGQ | |
| | | NYYGMDVWGQGTTVTVSS | |
| | HCDR1 | FTFSSYSMN | 58 |
| | HCDR2 | WVSSISSSSSYIYY | 59 |
| | HCDR3 | RDYRGYSGQNYYGMDV | 60 |
| | VL | QSALTQPPSVSAAPRQKVTISCSGSSSNIGNNDVSWYQQLPGTVPKLLIYDNN | 61 |
| | | KRPSGIPDRFSGSKSGTSASLGITGLQTGDEADYYCGVWDSSLSGYVFGTGTK | |
| | | VTVL | |
| | LCDR1 | SSNIGNNDVS | 62 |
| | LCDR2 | LLIYDNNKRPS | 63 |
| | LCDR3 | GVWDSSLSGY | 64 |
| 2020EP032- | VH | QVQLVESGGGLVKPGGSLRLSCAASGFSFNNAWMSWVRQAPGKGLEWVGRIKS | 65 |
| C09 | | KTDDATTEYAAPVEGRFTISRDDSKNMLYLQMDSLKTEDTAVYYCTTDLRWEE | |
| | | LPMYWGQGTLVTVSS | |
| | HCDR1 | FSENNAWMS | 66 |
| | HCDR2 | WVGRIKSKTDDATTEY | 67 |
| | HCDR3 | TDLRWEELPMY | 68 |
| | VL | VIWMTQSPSSLSASVGDRVTITCRASQGIRNDLGWYQQKPGKAPKVLIYAASS | 69 |
| | | LQSGVPSRFSGSGSGTDFTLTISSLQPEDFATYYCLQDYNYPQTFGQGTKVGI | |
| | | KR | |
| | LCDR1 | QGIRNDLG | 70 |
| | LCDR2 | VLIYAASSLQS | 71 |
| | LCDR3 | LQDYNYPQ | 72 |
| 2020EP083- | VH | EVQLVESGGGLVEPGGSLRLSCAASGFTFSSYSMSWVRQAPGKGLEWVGRIKS | 73 |
| G11 | | ETDGGTTDYAGPAKGKFIISRNDAENTVSLQMNSLKFEDTAVYHCTTDSSSSW | |
| | | FSYSFDNWGQETLVTVSS | |
| | HCDR1 | FTFSSYSMS | 74 |
| | HCDR2 | WVGRIKSETDGGTTDY | 75 |
| | HCDR3 | TDSSSSWFSYSFDN | 76 |
| | VL | EIVMTQSPATLSVSPGERATLSCRASQNVNSNLAWYQQKTGQAPRLLIFGAST | 77 |
| | | RATGIPARFSGSGSGTEFTLTIGSLQSEDFAVYYCQQYNNWPPTFGGWTKVEI | |
| | | K | |
| | LCDR1 | QNVNSNLA | 78 |
| | LCDR2 | LLIFGASTRAT | 79 |
| | LCDR3 | QQYNNWPP | 80 |
| 2020EP083- | VH | QLQLQESGPGLVKPAETLSLTCTVSGGSVSSGDNLWDWIRQPPGKGLEWIGSI | 81 |
| H01 | | RDSGNPAYNPSLRSRVTISADTSTDQFSLKLTSVTAADTAVYYCARHSDGGYG | |
| | | KIYGMDVWGQGTTVTVSS | |
| | HCDR1 | GSVSSGDNLWD | 82 |
| | HCDR2 | WIGSIRDSGNPAY | 83 |
| | HCDR3 | RHSDGGYGKIYGMDV | 84 |
| | VL | DIQLTQSPSFLSASVGDRVTITCRASQAVSTYLVWYLQKPGQAPRLLIYAAST | 85 |
| | | LQSGVPSRFSGSGSGTDFTLTISRLQPEDAGTYFCQQLNSNALVFGGGTKVDI | |
| | | K | |
| | LCDR1 | QAVSTYLV | 86 |
| | LCDR2 | LLIYAASTLQS | 87 |
| | LCDR3 | QQLNSNALV | 88 |
| 2020EP085- | VH | QVQLVQSGAEVKKPGASVKVSCKASGYTFTDYYMHWVRQAPGQGLEWMGWINP | 89 |
| G5 | | NSGGTNYAQKFQGRVTMTRDTSITTAYMELSWLRSDDTAVYYCAREVGTTTTG | |
| | | VDYWGQGTLVTVSS | |
| | HCDR1 | YTFTDYYMH | 90 |
| | HCDR2 | WMGWINPNSGGTNYA | 91 |
| | HCDR3 | REVGTTTTGVDY | 92 |
| | VL | VIWMTQSPSSLSASVGDRVTFTCQASQDINIYLTWYQIKPGKAPKVLIYDAST | 93 |
| | | LESGVPSRFSGSGFGTHFVFTISSLQPEDFATYYCQHYDSLPLTFGGGTKVEI | |
| | | K | |
| | LCDR1 | QDINIYLT | 94 |
| | LCDR2 | VLIYDASTLES | 95 |
| | LCDR3 | QHYDSLPL | 96 |

In some embodiments, the anti-Siglec15 antibodies described herein bind to the same epitope of a Siglec15 polypeptide as any of the exemplary antibodies described herein (for example, 2019EP47-A02, 2019EP47-A05, 2019EP47-A10, 2019EP47-C12, 2020EP032-A08, 2020EP032-A12, 2020EP032-B03, 2020EP032-H11, 2020EP032-C09, 2020EP083-G11, 2020EP083-H01, or 2020EP085-G5) or compete against the exemplary antibody from binding to the Siglec15 antigen. In some examples, the exemplary antibody is 2020EP032-H11 (a.k.a., H11). In other examples, the exemplary antibody is 2019EP47-A02. In yet other examples, the exemplary antibody is 2020EP032-B03.

An “epitope” refers to the site on a target antigen that is recognized and bound by an antibody. The site can be entirely composed of amino acid components, entirely composed of chemical modifications of amino acids of the protein (e.g., glycosyl moieties), or composed of combinations thereof. Overlapping epitopes include at least one common amino acid residue. An epitope can be linear, which is typically 6-15 amino acids in length. Alternatively, the epitope can be conformational. The epitope to which an antibody binds can be determined by routine technology, for example, the epitope mapping method (see, e.g., descriptions below). An antibody that binds the same epitope as an exemplary antibody described herein may bind to exactly the same epitope or a substantially overlapping epitope (e.g., containing less than 3 non-overlapping amino acid residues, less than 2 non-overlapping amino acid residues, or only 1 non-overlapping amino acid residue) as the exemplary antibody. Whether two antibodies compete against each other from binding to the cognate antigen can be determined by a competition assay, which is well known in the art.

In some examples, the anti-Siglec15 antibody comprises the same V_{H} and/or V_{L} CDRs as an exemplary antibody described herein. Two antibodies having the same V_{H} and/or V_{L} CDRs means that their CDRs are identical when determined by the same approach (e.g., the Kabat approach, the Chothia approach, the AbM approach, the Contact approach, or the IMGT approach as known in the art. See, e.g., bioinf.org.uk/abs/). Such anti-Siglec15 antibodies may have the same V_{H}, the same V_{L}, or both as compared to an exemplary antibody described herein.

Also within the scope of the present disclosure are functional variants of any of the exemplary anti-Siglec 15 antibodies as disclosed herein. Such functional variants are substantially similar to the exemplary antibody, both structurally and functionally. A functional variant comprises substantially the same V_{H} and V_{L} CDRs as the exemplary antibody. For example, it may comprise only up to 8 (e.g., 8, 7, 6, 5, 4, 3, 2, or 1) amino acid residue variations in the total CDR regions of the antibody and binds the same epitope of Siglec15 with substantially similar affinity (e.g., having a K_{D} value in the same order). In some instances, the functional variants may have the same heavy chain CDR3 as the exemplary antibody, and optionally the same light chain CDR3 as the exemplary antibody.

Alternatively or in addition, the functional variants may have the same heavy chain CDR2 as the exemplary antibody. Such an anti-Siglec 15 antibody may comprise a V_{H} fragment having CDR amino acid residue variations in only the heavy chain CDR1 as compared with the V_{H} of the exemplary antibody. In some examples, the anti-Siglec15 antibody may further comprise a V_{L} fragment having the same V_{L} CDR3, and optionally same V_{L} CDR1 or V_{L} CDR₂ as the exemplary antibody.

Alternatively or in addition, the amino acid residue variations can be conservative amino acid residue substitutions. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g., Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

In some embodiments, the anti-Siglec15 antibody may comprise heavy chain CDRs that are at least 80% (e.g., 85%, 90%, 95%, or 98%) sequence identity, individually or collectively, as compared with the V_{H} CDRs of an exemplary antibody described herein (e.g., H11). Alternatively or in addition, the anti-Siglec15 antibody may comprise light chain CDRs that are at least 80% (e.g., 85%, 90%, 95%, or 98%) sequence identity, individually or collectively, as compared with the V_{L} CDRs as an exemplary antibody described herein. As used herein, “individually” means that one CDR of an antibody shares the indicated sequence identity relative to the corresponding CDR of the exemplary antibody. “Collectively” means that three V_{H} or V_{L} CDRs of an antibody in combination share the indicated sequence identity relative the corresponding three V_{H} or V_{L} CDRs of the exemplary antibody in combination.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of interest. Where gaps exist between two sequences, Gapped BLAST can be utilized as described in Altschul et al., Nucleic Acids Res. 25 (17): 3389-3402, 1997. When utilizing BLAST and Gapped BLAST programs, the default parameters of the respective programs (e.g., XBLAST and NBLAST) can be used.

In some embodiments, the heavy chain of any of the anti-Siglec15 antibodies as described herein may further comprise a heavy chain constant region (CH) or a portion thereof (e.g., CH1, CH2, CH3, or a combination thereof). The heavy chain constant region can of any suitable origin, e.g., human, mouse, rat, or rabbit. Alternatively or in addition, the light chain of the anti-Siglec15 antibody may further comprise a light chain constant region (CL), which can be any CL known in the art. In some examples, the CL is a kappa light chain. In other examples, the CL is a lambda light chain. Antibody heavy and light chain constant regions are well known in the art, e.g., those provided in the IMGT database (www.imgt.org) or at www.vbase2.org/vbstat.php., both of which are incorporated by reference herein.

In some embodiments, the anti-Siglec15 antibody disclosed herein may be a single chain antibody (scFv). A scFv antibody may comprise a V_{H} fragment and a V_{L} fragment, which may be linked via a flexible peptide linker. In some instances, the scFv antibody may be in the V_{H}→V_{L} orientation (from N-terminus to C-terminus). In other instances, the scFv antibody may be in the V_{L}→V_{H} orientation (from N-terminus to C-terminus).

Any of the anti-Siglec15 antibody as described herein, e.g., the exemplary anti-Siglec15 antibodies provided here, can bind and inhibit (e.g., reduce or eliminate) the activity of Siglec15-positive cells (e.g., immune cells or cancer cells). In some embodiments, the anti-Siglec15 antibody as described herein can bind and inhibit the activity of Siglec15-positive cells by at least 30% (e.g., 35%, 40%, 45%, 50%, 60%, 70%, 80%, 90%, 95% or greater, including any increment therein). The inhibitory activity of an anti-Siglec 15 antibody described herein can be determined by routine methods known in the art, e.g., by an assay for measuring the K_{i,}^{app} value.

In some examples, the K_{i,}^{app} value of an antibody may be determined by measuring the inhibitory effect of different concentrations of the antibody on the extent of a relevant reaction; fitting the change in pseudo-first order rate constant (v) as a function of inhibitor concentration to the modified Morrison equation (Equation 1) yields an estimate of the apparent Ki value. For a competitive inhibitor, the Ki^{app} can be obtained from the y-intercept extracted from a linear regression analysis of a plot of K_{i,}^{app} versus substrate concentration.

$$v = A \cdot \frac{( [ E ] - [ I ] - {K}_{i}^{a p p} ) + \sqrt{{( [ E ] - [ I ] - {K}_{i}^{a p p} )}^{2} + 4 [ E ] \cdot } {K}_{i}^{a p p}}{2} \qquad ( Equation 1 )$$

Where A is equivalent to vₒ/E, the initial velocity (vₒ) of the enzymatic reaction in the absence of inhibitor (I) divided by the total enzyme concentration (E). In some embodiments, the anti-Siglec15 antibody described herein may have a Ki^{app} value of 1000, 500, 100, 50, 40, 30, 20, 10, 5 pM or less for the target antigen or antigen epitope.

### II. Preparation of Anti-Siglec15 Antibodies

Antibodies capable of binding Siglec15 such as human Siglec15 as described herein can be made by any method known in the art. See, for example, Harlow and Lane, (1998) Antibodies: A Laboratory Manual, Cold Spring Harbor Laboratory, New York. In some embodiments, the antibody may be produced by the conventional hybridoma technology. Alternatively, the anti-Siglec 15 antibody may be identified from a suitable library (e.g., a human antibody library).

In some instances, high affinity fully human Siglec 15 binders may be obtained from a human antibody library following the screening strategy described in Example 1 below. This strategy allows for maximizing the library diversity to cover board and active epitopes on Siglec15-expressing cells.

If desired, an antibody (monoclonal or polyclonal) of interest (e.g., produced by a hybridoma cell line or isolated from an antibody library) may be sequenced and the polynucleotide sequence may then be cloned into a vector for expression or propagation. The sequence encoding the antibody of interest may be maintained in vector in a host cell and the host cell can then be expanded and frozen for future use. In an alternative, the polynucleotide sequence may be used for genetic manipulation to, e.g., humanize the antibody or to improve the affinity (affinity maturation), or other characteristics of the antibody. For example, the constant region may be engineered to more resemble human constant regions to avoid immune response if the antibody is from a non-human source and is to be used in clinical trials and treatments in humans. Alternatively or in addition, it may be desirable to genetically manipulate the antibody sequence to obtain greater affinity and/or specificity to the target antigen and greater efficacy in enhancing the activity of Siglec15. It will be apparent to one of skill in the art that one or more polynucleotide changes can be made to the antibody and still maintain its binding specificity to the target antigen.

Alternatively, antibodies capable of binding to the target antigens as described herein (a Siglec 15 molecule such as a human Siglec15) may be isolated from a suitable antibody library via routine practice. Antibody libraries can be used to identify proteins that bind to a target antigen (e.g., human Siglec15 such as cell surface Siglec 15) via routine screening processes. In the selection process, the polypeptide component is probed with the target antigen or a fragment thereof and, if the polypeptide component binds to the target, the antibody library member is identified, typically by retention on a support. Retained display library members are recovered from the support and analyzed. The analysis can include amplification and a subsequent selection under similar or dissimilar conditions. For example, positive and negative selections can be alternated. The analysis can also include determining the amino acid sequence of the polypeptide component and purification of the polypeptide component for detailed characterization.

There are a number of routine methods known in the art to identify and isolate antibodies capable of binding to the target antigens described herein, including phage display, yeast display, ribosomal display, or mammalian display technology.

Antigen-binding fragments of an intact antibody (full-length antibody) can be prepared via routine methods. For example, F(ab′)2 fragments can be produced by pepsin digestion of an antibody molecule, and Fab fragments that can be generated by reducing the disulfide bridges of F(ab′)2 fragments.

Genetically engineered antibodies, such as humanized antibodies, chimeric antibodies, single-chain antibodies, and bi-specific antibodies, can be produced via, e.g., conventional recombinant technology. In one example, DNA encoding a monoclonal antibodies specific to a target antigen can be readily isolated and sequenced using conventional procedures (e.g., by using oligonucleotide probes that are capable of binding specifically to genes encoding the heavy and light chains of the monoclonal antibodies). Once isolated, the DNA may be placed into one or more expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. See, e.g., PCT Publication No. WO 87/04462. The DNA can then be modified, for example, by substituting the coding sequence for human heavy and light chain constant domains in place of the homologous murine sequences, Morrison et al., (1984) Proc. Nat. Acad. Sci. 81:6851, or by covalently joining to the immunoglobulin coding sequence all or part of the coding sequence for a non-immunoglobulin polypeptide. In that manner, genetically engineered antibodies, such as “chimeric” or “hybrid” antibodies; can be prepared that have the binding specificity of a target antigen.

Techniques developed for the production of “chimeric antibodies” are well known in the art. See, e.g., Morrison et al. (1984) Proc. Natl. Acad. Sci. USA 81, 6851; Neuberger et al. (1984) Nature 312, 604; and Takeda et al. (1984) Nature 314:452.

Methods for constructing humanized antibodies are also well known in the art. See, e.g., Queen et al., Proc. Natl. Acad. Sci. USA, 86:10029-10033 (1989). In one example, variable regions of V_{H} and V_{L} of a parent non-human antibody are subjected to three-dimensional molecular modeling analysis following methods known in the art. Next, framework amino acid residues predicted to be important for the formation of the correct CDR structures are identified using the same molecular modeling analysis. In parallel, human V_{H} and V_{L} chains having amino acid sequences that are homologous to those of the parent non-human antibody are identified from any antibody gene database using the parent V_{H} and V_{L} sequences as search queries. Human V_{H} and V_{L} acceptor genes are then selected.

The CDR regions within the selected human acceptor genes can be replaced with the CDR regions from the parent non-human antibody or functional variants thereof. When necessary, residues within the framework regions of the parent chain that are predicted to be important in interacting with the CDR regions (see above description) can be used to substitute for the corresponding residues in the human acceptor genes.

A single-chain antibody can be prepared via recombinant technology by linking a nucleotide sequence coding for a heavy chain variable region and a nucleotide sequence coding for a light chain variable region. Preferably, a flexible linker is incorporated between the two variable regions. Alternatively, techniques described for the production of single chain antibodies (U.S. Pat. Nos. 4,946,778 and 4,704,692) can be adapted to produce a phage-display, yeast-display, mammalian cell-display, or mRNA-display scFv library and scFv clones specific to Siglec 15 can be identified from the library following routine procedures. Positive clones can be subjected to further screening to identify those that binds the Siglec 15 antigen.

Antibodies obtained following a method known in the art and described herein can be characterized using methods well known in the art. For example, one method is to identify the epitope to which the antigen binds, or “epitope mapping.” There are many methods known in the art for mapping and characterizing the location of epitopes on proteins, including solving the crystal structure of an antibody-antigen complex, competition assays, gene fragment expression assays, and synthetic peptide-based assays, as described, for example, in Chapter 11 of Harlow and Lane, Using Antibodies, a Laboratory Manual, Cold Spring Harbor Laboratory Press, Cold Spring Harbor, N.Y., 1999. In an additional example, epitope mapping can be used to determine the sequence, to which an antibody binds. The epitope can be a linear epitope, i.e., contained in a single stretch of amino acids, or a conformational epitope formed by a three-dimensional interaction of amino acids that may not necessarily be contained in a single stretch (primary structure linear sequence). Peptides of varying lengths (e.g., at least 4-6 amino acids long) can be isolated or synthesized (e.g., recombinantly) and used for binding assays with an antibody. In another example, the epitope to which the antibody binds can be determined in a systematic screening by using overlapping peptides derived from the target antigen sequence and determining binding by the antibody. According to the gene fragment expression assays, the open reading frame encoding the target antigen is fragmented either randomly or by specific genetic constructions and the reactivity of the expressed fragments of the antigen with the antibody to be tested is determined. The gene fragments may, for example, be produced by PCR and then transcribed and translated into protein in vitro, in the presence of radioactive amino acids. The binding of the antibody to the radioactively labeled antigen fragments is then determined by immunoprecipitation and gel electrophoresis. Certain epitopes can also be identified by using large libraries of random peptide sequences displayed on the surface of phage particles (phage libraries).

Alternatively, a defined library of overlapping peptide fragments can be tested for binding to the test antibody in simple binding assays. In an additional example, mutagenesis of an antigen binding domain, domain swapping experiments and alanine scanning mutagenesis can be performed to identify residues required, sufficient, and/or necessary for epitope binding. For example, domain swapping experiments can be performed using a mutant of a target antigen in which various fragments of Siglec 15 have been replaced (swapped) with sequences from a closely related, but antigenically distinct protein (such as another member of the tumor necrosis factor receptor family). By assessing binding of the antibody to the mutant Siglec 15, the importance of the particular antigen fragment to antibody binding can be assessed.

Alternatively, competition assays can be performed using other antibodies known to bind to the same antigen to determine whether an antibody binds to the same epitope as the other antibodies. Competition assays are well known to those of skill in the art.

In some examples, an anti-Siglec15 antibody is prepared by recombinant technology as exemplified below.

Nucleic acids encoding the heavy and light chain of an anti-Siglec15 antibody as described herein can be cloned into one expression vector, each nucleotide sequence being in operable linkage to a suitable promoter. In one example, each of the nucleotide sequences encoding the heavy chain and light chain is in operable linkage to a distinct prompter. Alternatively, the nucleotide sequences encoding the heavy chain and the light chain can be in operable linkage with a single promoter, such that both heavy and light chains are expressed from the same promoter. When necessary, an internal ribosomal entry site (IRES) can be inserted between the heavy chain and light chain encoding sequences.

In some examples, the nucleotide sequences encoding the two chains of the antibody are cloned into two vectors, which can be introduced into the same or different cells. When the two chains are expressed in different cells, each of them can be isolated from the host cells expressing such and the isolated heavy chains and light chains can be mixed and incubated under suitable conditions allowing for the formation of the antibody.

Generally, a nucleic acid sequence encoding one or all chains of an antibody can be cloned into a suitable expression vector in operable linkage with a suitable promoter using methods known in the art. For example, the nucleotide sequence and vector can be contacted, under suitable conditions, with a restriction enzyme to create complementary ends on each molecule that can pair with each other and be joined together with a ligase. Alternatively, synthetic nucleic acid linkers can be ligated to the termini of a gene. These synthetic linkers contain nucleic acid sequences that correspond to a particular restriction site in the vector. The selection of expression vectors/promoter would depend on the type of host cells for use in producing the antibodies.

A variety of promoters can be used for expression of the antibodies described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV5 promoter, and the herpes simplex tk virus promoter.

Regulatable promoters can also be used. Such regulatable promoters include those using the lac repressor from E. coli as a transcription modulator to regulate transcription from lac operator-bearing mammalian cell promoters [Brown, M. et al., Cell, 49:603-612 (1987)], those using the tetracycline repressor (tetR) [Gossen, M., and Bujard, H., Proc. Natl. Acad. Sci. USA 89:5547-5551 (1992); Yao, F. et al., Human Gene Therapy, 9:1939-1950 (1998); Shockelt, P., et al., Proc. Natl. Acad. Sci. USA, 92:6522-6526 (1995)]. Other systems include FK506 dimer, VP16 or p65 using astradiol, RU486, diphenol murislerone, or rapamycin. Inducible systems are available from Invitrogen, Clontech and Ariad.

Regulatable promoters that include a repressor with the operon can be used. In one embodiment, the lac repressor from E. coli can function as a transcriptional modulator to regulate transcription from lac operator-bearing mammalian cell promoters [M. Brown et al., Cell, 49:603-612 (1987); Gossen and Bujard (1992); M. Gossen et al., Natl. Acad. Sci. USA, 89:5547-5551 (1992)] combined the tetracycline repressor (tetR) with the transcription activator (VP 16) to create a tetR-mammalian cell transcription activator fusion protein, tTa (tetR-VP 16), with the tetO-bearing minimal promoter derived from the human cytomegalovirus (hCMV) major immediate-early promoter to create a tetR-tet operator system to control gene expression in mammalian cells. In one embodiment, a tetracycline inducible switch is used. The tetracycline repressor (tetR) alone, rather than the tetR-mammalian cell transcription factor fusion derivatives can function as potent trans-modulator to regulate gene expression in mammalian cells when the tetracycline operator is properly positioned downstream for the TATA element of the CMVIE promoter (Yao et al., Human Gene Therapy, 10 (16): 1392-1399 (2003)). One particular advantage of this tetracycline inducible switch is that it does not require the use of a tetracycline repressor-mammalian cells transactivator or repressor fusion protein, which in some instances can be toxic to cells (Gossen et al., Natl. Acad. Sci. USA, 89:5547-5551 (1992); Shockett et al., Proc. Natl. Acad. Sci. USA, 92:6522-6526 (1995)), to achieve its regulatable effects.

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art.

Examples of polyadenylation signals useful to practice the methods described herein include, but are not limited to, human collagen I polyadenylation signal, human collagen II polyadenylation signal, and SV40 polyadenylation signal.

One or more vectors (e.g., expression vectors) comprising nucleic acids encoding any of the antibodies may be introduced into suitable host cells for producing the antibodies. The host cells can be cultured under suitable conditions for expression of the antibody or any polypeptide chain thereof. Such antibodies or polypeptide chains thereof can be recovered by the cultured cells (e.g., from the cells or the culture supernatant) via a conventional method, e.g., affinity purification. If necessary, polypeptide chains of the antibody can be incubated under suitable conditions for a suitable period of time allowing for production of the antibody.

In some embodiments, methods for preparing an antibody described herein involve a recombinant expression vector that encodes both the heavy chain and the light chain of an anti-Siglec15 antibody, as also described herein. The recombinant expression vector can be introduced into a suitable host cell (e.g., a dhfr-CHO cell) by a conventional method, e.g., calcium phosphate-mediated transfection. Positive transformant host cells can be selected and cultured under suitable conditions allowing for the expression of the two polypeptide chains that form the antibody, which can be recovered from the cells or from the culture medium. When necessary, the two chains recovered from the host cells can be incubated under suitable conditions allowing for the formation of the antibody.

In one example, two recombinant expression vectors are provided, one encoding the heavy chain of the anti-Siglec 15 antibody and the other encoding the light chain of the anti-Siglec 15 antibody. Both of the two recombinant expression vectors can be introduced into a suitable host cell (e.g., dhfr-CHO cell) by a conventional method, e.g., calcium phosphate-mediated transfection. Alternatively, each of the expression vectors can be introduced into a suitable host cells. Positive transformants can be selected and cultured under suitable conditions allowing for the expression of the polypeptide chains of the antibody. When the two expression vectors are introduced into the same host cells, the antibody produced therein can be recovered from the host cells or from the culture medium. If necessary, the polypeptide chains can be recovered from the host cells or from the culture medium and then incubated under suitable conditions allowing for formation of the antibody. When the two expression vectors are introduced into different host cells, each of them can be recovered from the corresponding host cells or from the corresponding culture media. The two polypeptide chains can then be incubated under suitable conditions for formation of the antibody.

Standard molecular biology techniques are used to prepare the recombinant expression vector, transfect the host cells, select for transformants, culture the host cells and recovery of the antibodies from the culture medium. For example, some antibodies can be isolated by affinity chromatography with a Protein A or Protein G coupled matrix.

Any of the nucleic acids encoding the heavy chain, the light chain, or both of an anti-Siglec15 antibody as described herein, vectors (e.g., expression vectors) containing such; and host cells comprising the vectors are within the scope of the present disclosure.

### III. Applications of Anti-Siglec15 Antibodies

Any of the anti-Siglec 15 antibodies disclosed herein can be used for therapeutic, diagnostic, and/or research purposes, all of which are within the scope of the present disclosure.

### Pharmaceutical Compositions

The antibodies, as well as the encoding nucleic acids or nucleic acid sets, vectors comprising such, or host cells comprising the vectors, as described herein can be mixed with a pharmaceutically acceptable carrier (excipient) to form a pharmaceutical composition for use in treating a target disease. “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover.

The pharmaceutical compositions to be used in the present methods can comprise pharmaceutically acceptable carriers, excipients, or stabilizers in the form of lyophilized formulations or aqueous solutions. Remington: The Science and Practice of Pharmacy 20th Ed. (2000) Lippincott Williams and Wilkins, Ed. K. E. Hoover. Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

In some examples, the pharmaceutical composition described herein comprises liposomes containing the antibodies (or the encoding nucleic acids) which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The antibodies, or the encoding nucleic acid(s), may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are known in the art, see, e.g., Remington, The Science and Practice of Pharmacy 20th Ed. Mack Publishing (2000).

In other examples, the pharmaceutical composition described herein can be formulated in sustained-release format. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly (2-hydroxyethyl-methacrylate), or poly (vinyl alcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The pharmaceutical compositions to be used for in vivo administration must be sterile. This is readily accomplished by, for example, filtration through sterile filtration membranes. Therapeutic antibody compositions are generally placed into a container having a sterile access port, for example, an intravenous solution bag or vial having a stopper pierceable by a hypodermic injection needle.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g., Tween™ 20, 40, 60, 80 or 85) and other sorbitans (e.g., Span™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets between 0.1 and 1.0 μm, particularly 0.1 and 0.5 μm, and have a pH in the range of 5.5 to 8.0.

The emulsion compositions can be those prepared by mixing an antibody with Intralipid™ or the components thereof (soybean oil, egg phospholipids, glycerol and water).

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

### Therapeutic Applications

To practice the method disclosed herein, an effective amount of the pharmaceutical composition described herein can be administered to a subject (e.g., a human) in need of the treatment via a suitable route, such as intravenous administration, e.g., as a bolus or by continuous infusion over a period of time, by intramuscular, intraperitoneal, intracerebrospinal, subcutaneous, intra-articular, intrasynovial, intrathecal, oral, inhalation or topical routes. Commercially available nebulizers for liquid formulations, including jet nebulizers and ultrasonic nebulizers are useful for administration. Liquid formulations can be directly nebulized and lyophilized powder can be nebulized after reconstitution. Alternatively, the antibodies as described herein can be aerosolized using a fluorocarbon formulation and a metered dose inhaler, or inhaled as a lyophilized and milled powder.

The subject to be treated by the methods described herein can be a mammal, more preferably a human. Mammals include, but are not limited to, farm animals, sport animals, pets, primates, horses, dogs, cats, mice and rats. A human subject who needs the treatment may be a human patient having, at risk for, or suspected of having a target disease/disorder characterized by carrying Siglec15⁺ disease cells. Examples of such target diseases/disorders include cancer, immunological disorders (e.g., autoimmune diseases), and osteoporosis. Exemplary cancers include, but are not limited to, non-small cell lung cancer (NSCLC), ovarian cancer, breast cancer, head-and-neck cancer, renal carcinoma, pancreatic cancer, endometrial cancer, urothelial cancer, thyroid cancer, colon cancer, colorectal cancer, melanoma, liver cancer, and gastric cancer.

A subject having a target cancer can be identified by routine medical examination, e.g., laboratory tests, organ functional tests, CT scans, or ultrasounds. In some embodiments, the subject to be treated by the method described herein may be a human cancer patient who has undergone or is subjecting to an anti-cancer therapy, for example, chemotherapy, radiotherapy, immunotherapy, or surgery.

A subject suspected of having any of such target disease/disorder might show one or more symptoms of the disease/disorder. A subject at risk for the disease/disorder can be a subject having one or more of the risk factors for that disease/disorder.

As used herein, “an effective amount” refers to the amount of each active agent required to confer therapeutic effect on the subject, either alone or in combination with one or more other active agents. Determination of whether an amount of the antibody achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

Empirical considerations, such as the half-life, generally will contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of a target disease/disorder. Alternatively, sustained continuous release formulations of an antibody may be appropriate. Various formulations and devices for achieving sustained release are known in the art.

In one example, dosages for an antibody as described herein may be determined empirically in individuals who have been given one or more administration(s) of the antibody. Individuals are given incremental dosages of the agonist. To assess efficacy of the agonist, an indicator of the disease/disorder can be followed.

Generally, for administration of any of the antibodies described herein, an initial candidate dosage can be about 2 mg/kg. For the purpose of the present disclosure, a typical daily dosage might range from about any of 0.1 μg/kg to 3 μg/kg to 30 μg/kg to 300 μg/kg to 3 mg/kg, to 30 mg/kg to 100 mg/kg or more, depending on the factors mentioned above. For repeated administrations over several days or longer, depending on the condition, the treatment is sustained until a desired suppression of symptoms occurs or until sufficient therapeutic levels are achieved to alleviate a target disease or disorder, or a symptom thereof. An exemplary dosing regimen comprises administering an initial dose of about 2 mg/kg, followed by a weekly maintenance dose of about 1 mg/kg of the antibody, or followed by a maintenance dose of about 1 mg/kg every other week. However, other dosage regimens may be useful, depending on the pattern of pharmacokinetic decay that the practitioner wishes to achieve. For example, dosing from one-four times a week is contemplated. In some embodiments, dosing ranging from about 3 μg/mg to about 2 mg/kg (such as about 3 μg/mg, about 10 μg/mg, about 30 μg/mg, about 100 μg/mg, about 300 μg/mg, about 1 mg/kg, and about 2 mg/kg) may be used. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen (including the antibody used) can vary over time.

In some embodiments, for an adult patient of normal weight, doses ranging from about 0.3 to 5.00 mg/kg may be administered. In some examples, the dosage of the anti-Siglec 15 antibody described herein can be 10 mg/kg. The particular dosage regimen, i.e., dose, timing and repetition, will depend on the particular individual and that individual's medical history, as well as the properties of the individual agents (such as the half-life of the agent, and other considerations well known in the art).

For the purpose of the present disclosure, the appropriate dosage of an antibody as described herein will depend on the specific antibody, antibodies, and/or non-antibody peptide (or compositions thereof) employed, the type and severity of the disease/disorder, whether the antibody is administered for preventive or therapeutic purposes, previous therapy, the patient's clinical history and response to the agonist, and the discretion of the attending physician. Typically the clinician will administer an antibody, until a dosage is reached that achieves the desired result. In some embodiments, the desired result is an increase in anti-tumor immune response in the tumor microenvironment. Methods of determining whether a dosage resulted in the desired result would be evident to one of skill in the art. Administration of one or more antibodies can be continuous or intermittent, depending, for example, upon the recipient's physiological condition, whether the purpose of the administration is therapeutic or prophylactic, and other factors known to skilled practitioners. The administration of an antibody may be essentially continuous over a preselected period of time or may be in a series of spaced dose, e.g., either before, during, or after developing a target disease or disorder.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

Alleviating a target disease/disorder includes delaying the development or progression of the disease, or reducing disease severity or prolonging survival. Alleviating the disease or prolonging survival does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

Conventional methods, known to those of ordinary skill in the art of medicine, can be used to administer the pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods. In some examples, the pharmaceutical composition is administered intraocularly or intravitreally.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and a physiologically acceptable excipient is infused. Physiologically acceptable excipients may include, for example, 5% dextrose, 0.9% saline, Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

In one embodiment, an antibody is administered via site-specific or targeted local delivery techniques. Examples of site-specific or targeted local delivery techniques include various implantable depot sources of the antibody or local delivery catheters, such as infusion catheters, an indwelling catheter, or a needle catheter, synthetic grafts, adventitial wraps, shunts and stents or other implantable devices, site specific carriers, direct injection, or direct application. See, e.g., PCT Publication No. WO 00/53211 and U.S. Pat. No. 5,981,568.

Targeted delivery of therapeutic compositions containing an antisense polynucleotide, expression vector, or subgenomic polynucleotides can also be used. Receptor-mediated DNA delivery techniques are described in, for example, Findeis et al., Trends Biotechnol. (1993) 11:202; Chiou et al., Gene Therapeutics: Methods And Applications Of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. USA (1990) 87:3655; Wu et al., J. Biol. Chem. (1991) 266:338.

Therapeutic compositions containing a polynucleotide (e.g., those encoding the antibodies described herein) are administered in a range of about 100 ng to about 200 mg of DNA for local administration in a gene therapy protocol. In some embodiments, concentration ranges of about 500 ng to about 50 mg, about 1 μg to about 2 mg, about 5 μg to about 500 μg, and about 20 μg to about 100 μg of DNA or more can also be used during a gene therapy protocol.

The therapeutic polynucleotides and polypeptides described herein can be delivered using gene delivery vehicles. The gene delivery vehicle can be of viral or non-viral origin (see generally, Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). Expression of such coding sequences can be induced using endogenous mammalian or heterologous promoters and/or enhancers. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5,219,740 and 4,777,127; GB Patent No. 2,200,651; and EP Patent No. 0 345 242), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors (see, e.g., PCT Publication Nos. WO 94/12649, WO 93/03769; WO 93/19191; WO 94/28938; WO 95/11984 and WO 95/00655). Administration of DNA linked to killed adenovirus as described in Curiel, Hum. Gene Ther. (1992) 3:147 can also be employed.

Non-viral delivery vehicles and methods can also be employed, including, but not limited to, polycationic condensed DNA linked or unlinked to killed adenovirus alone (see, e.g., Curiel, Hum. Gene Ther. (1992) 3:147); ligand-linked DNA (see, e.g., Wu, J. Biol. Chem. (1989) 264:16985); eukaryotic cell delivery vehicles cells (see, e.g., U.S. Pat. No. 5,814,482; PCT Publication Nos. WO 95/07994; WO 96/17072; WO 95/30763; and WO 97/42338) and nucleic charge neutralization or fusion with cell membranes. Naked DNA can also be employed. Exemplary naked DNA introduction methods are described in PCT Publication No. WO 90/11092 and U.S. Pat. No. 5,580,859. Liposomes that can act as gene delivery vehicles are described in U.S. Pat. No. 5,422,120; PCT Publication Nos. WO 95/13796; WO 94/23697; WO 91/14445; and EP U.S. Pat. No. 524,968. Additional approaches are described in Philip, Mol. Cell. Biol. (1994) 14:2411, and in Woffendin, Proc. Natl. Acad. Sci. (1994) 91:1581.

The particular dosage regimen, i.e., dose, timing and repetition, used in the method described herein will depend on the particular subject and that subject's medical history.

In some embodiments, more than one antibody, or a combination of an antibody and another suitable therapeutic agent, may be administered to a subject in need of the treatment. The antibody can also be used in conjunction with other agents that serve to enhance and/or complement the effectiveness of the agents.

Treatment efficacy for a target disease/disorder can be assessed by methods well-known in the art.

### Kits for Use in Treatment of Diseases

The present disclosure also provides kits for use in treating or alleviating a target disease, such as hematopoietic cancer as described herein. Such kits can include one or more containers comprising an anti-Siglec15 antibody, e.g., any of those described herein. In some instances, the anti-Siglec15 antibody may be co-used with a second therapeutic agent.

In some embodiments, the kit can comprise instructions for use in accordance with any of the methods described herein. The included instructions can comprise a description of administration of the anti-Siglec15 antibody, and optionally the second therapeutic agent, to treat, delay the onset, or alleviate a target disease as those described herein. The kit may further comprise a description of selecting an individual suitable for treatment based on identifying whether that individual has the target disease, e.g., applying the diagnostic method as described herein. In still other embodiments, the instructions comprise a description of administering an antibody to an individual at risk of the target disease.

The instructions relating to the use of an anti-Siglec15 antibody generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating the disease, such as cancer or immune disorders (e.g., an autoimmune disease). Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an anti-Siglec15 antibody as those described herein.

Kits may optionally provide additional components such as buffers and interpretive information. Normally, the kit comprises a container and a label or package insert(s) on or associated with the container. In some embodiments, the invention provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1. Discovery of Anti-Siglec15 Antibody

This example describes identification of exemplary anti-Siglec15 antibodies via the mRNA display technology.

### Siglec15 Recombinant Cell Line Generation

HEK293 cells (ATCC) were transfected with a construct encoding the full-length human Siglec15 with C-terminal flag and Myc tags in pCMV6-Entry vector. G418 drug selection process yielded a polyclonal, drug resistant pool of Siglec15 target-expressing cells. In parallel, the empty vector transfected parental line was generated as a negative control. The Siglec15 target-expressing cells were sorted by FACS to yield a Siglec15 target expressing polyclonal pool. The pool was expanded under G418 drug selection. Single cell sorting then was performed followed by further drug selection to form clonal cell lines. The clonal lines were screened for Siglec 15 expression by FACS. The high expression Siglec 15 cell line was then used for selection and screening assays.

Anti-Siglec15 Single Chain Variable Fragment (scFv) Antibody Selection by mRNA Display

mRNA display technology was used for the identification of Siglec 15 binders from 10¹²⁻¹³ natural human scFv libraries. Briefly, the scFv DNA libraries were first transcribed into mRNA libraries and then translated into mRNA-scFv fusion libraries by covalent coupling through a puromycin linker, similar to the reported procedure (U.S. Pat. No. 6,258,558 B1, the relevant disclosures of which are incorporated by reference for the subject matter and purposes referenced herein). The fusion libraries were first counter selected with human IgGs (negative proteins) multiple times to remove non-specific binders, followed by selection against recombinant Siglec 15-Fc fusion protein (Acro #SG5-H5253), then captured on Protein G magnetic beads. Binders were then enriched by PCR amplification with library specific oliogs. At round 3-5, the scFv was also selected on recombinant Siglec 15/HEK cell lines in parallel. A total of 5 rounds of selections were executed to generate highly enriched Siglec15 binding pools for screening.

Identifications and Characterization of Anti-Siglec15 scFv Antibodies

After 5 rounds of selections, the Siglec15 enriched scFv libraries were cloned into bacterial periplasmic expression vector pET22b and transformed into TOP 10 competent cells. Each of the scFv molecule was engineered to have a C-terminal flag and 6× His tag for purification and assay detection. Clones from TOP 10 cells were pooled and the miniprep DNA were prepared and subsequently transformed into bacterial Rosetta II strain for expression. A single clone was picked, grown and induced with 0.1 mM IPTG in 96 well plate for expression. The supernatant was collected after 16-24 hours induction at 30° C. for assays to identify anti-Siglec 15 antibodies.

Siglec 15 binding screening ELISA was developed for the identification of individual anti-Siglec15 antibodies. Briefly, a 384 well plate was immobilized with human Fc, human Siglec15-Fc respectively, at a final concentration of 2 ug/mL in 1×PBS in a total volume of 25 uL per well. The plate was incubated overnight at 4° C. followed by blocking with 80 uL of superblock per well for 1 hour. 25 uL of supernatant was added to Fc and human Siglec15 immobilized wells and incubated for 1 hour with shaking. The Siglec 15 binding was detected by adding 25 uL of anti-Flag HRP diluted at 1:5000 in 1×PBST. In between each step, the plate was washed 3 times with 1×PBST in a plate washer. The plate was then developed with 20 uL of TMB substrate for 5 mins and stopped by adding 20 uL of 2 N sulfuric acid. The plate was read at OD450 nm Biotek plate reader and the binding and selectivity was analyzed with Excel bar graph. Clones with Siglec 15 target binding over human Fc>2-fold were subjected for DNA sequencing. The unique clones were produced and purified for further characterization.

ScFv Antibody Production in E. coli

The specified anti-Siglec 15 clone was picked from a glycerol stock plate and grown overnight into a 5 mL culture in a Thomson 24-well plate with a breathable membrane. This culture, and all subsequent cultures described below were grown at 37° C. and shaking at 225 RPM in Terrific Broth Complete plus 100 ug/mL carbenicillin and 34 ug/mL chloramphenicol, with 1:5,000 dilution of antifoam-204 also added, unless specified otherwise. This overnight starter culture was then used to inoculate the larger culture, 1:100 dilution of starter culture into the designated production culture and grown until OD600 was between 0.5-0.8. At this point, the culture was induced with a final concentration of IPTG at 0.1 mM and incubated over night at 30° C. The following day, the cultures were spun for 30 min at 5,000×g, to pellet the cells and then the supernatant was filter sterilized through a 0.2 um sterilizing PES membrane.

For the purification, 3 uL GE Ni Sepharose Excel resin per 1 mL of filtered supernatant was used. Disposable 10 mL or 20 mL BioRad Econo-Pac columns were used. The resin was equilibrated with at least 20 column volume (CV) buffer A (1×PBS, pH7.4 with extra NaCl added to 500 mM). The filter sterilized supernatant was purified by gravity flow by either controlling the flow to 1 mL/min or was poured over two times, over the same packed resin bed. The column was then washed with the following buffers: 10 CV buffer A, 20 CV buffer B (1×PBS, pH 7.4 with extra NaCl to 500 mM, and 30 mM imidazole). The two Detox buffers were used to remove endotoxin as optional step, if needed. For 250 mL expression culture purifications, the antibody-bound column was washed sequentially with 20 CV buffer C (1×PBS pH 7.4 with extra NaCl to 500 mM, 1% Tx114), 20 CV buffer D (1×PBS pH7.4 with extra NaCl to 500 mM, 1% Tx100+0.2% TNBP) and 40 CV buffer E (1×PBS pH7.4 with extra NaCl to 500 mM). The protein was eluted with Eluting buffer F (1×PBS pH 7.4 with extra NaCl to 500 mM, and 500 mM imidazole) in a total of six fractions (0.5 CV pre elute, 5×1 CV elute). Fractions were run on a Bradford assay (100 ul diluted Bradford solution+10 ul sample). Fractions with bright blue color were pooled and protein concentration was measured by A280 extension coefficient. SDS-PAGE gel was used to analyze the purity of the purified antibodies. In most cases, Tm shift thermal stability assay was run to measure the thermal stability of the purified antibodies.

### Determining Siglec15 Binding Via ELISA

An ELISA assay was developed to determine the EC50 of anti-Siglec15 antibodies. Briefly, a 384 well plate was immobilized with anti-human Fc antibody at final concentration of 2 ug/mL in 1×PBS in total volume of 25 uL per well. The plate was incubated overnight at 4° C. followed by blocking with 80 uL of superblock per well for 1 hour. Human Siglec 15-Fc was captured through immobilized anti-hFc antibody. Purified anti-Siglec 15 scFvs were 2-fold serial titrated from 200 nM. 25 uL was added to human Siglec15 immobilized wells and incubated for 1 hour with shaking. The Siglec15 binding was detected by adding 25 uL of anti-Flag HRP diluted at 1:5000 in 1×PBST. In between each step, the plate was washed 3 times with 1×PBST in a plate washer. The plate was then developed with 20 uL of TMB substrate for 5 mins and stopped by adding 20 uL of 2 N sulfuric acid. The plate was read at OD450 nm Biotek plate reader and then plotted in Prism 8.1 software. EC₅₀ values of exemplary anti-Siglec15 antibodies identified as disclosed herein were calculated and shown in Table 2 below.

TABLE 2
EC₅₀ Values of Exemplary Anti-Siglec15 Antibodies

| Exemplary Antibody Clones | EC50 (nM) |
|---|---|
| 2019EP47-A02 | 21.9 |
| 2019EP47-A05 | 3.355 |
| 2019EP47-A10 | 2.501 |
| 2019EP47-C12 | 9.973 |
| 2020EP032-A08 | 5.867 |
| 2020EP032-A12 | 0.544 |
| 2020EP032-B03 | 1.778 |
| 2020EP032-H11 | 0.5668 |
| 2020EP032-C09 | 6.073 |
| 2020EP083-G11 | 1.292 |
| 2020EP083-H01 | 2.544 |
| 2020EP085-G5 | 3.297 |

### Kinetic Analysis of ScFv Antibody Binding to Siglec 15 by SPR

Kinetic analysis of anti-Siglec15 scFvs were assessed by SPR technology with Biacore T200. The assay was run with Biacore T200 control software version 2.0. Protein A sensor chip was used to capture Fc fusion protein in the assay. For each cycle, 1 ug/mL of human Siglec15-Fc protein was captured for 60 seconds at flow rate of 10 ul/min on flow cell 2 in 1× HBSP buffer on Protein A sensor chip. 2-fold serial diluted HIS tag purified anti-Siglec15 scFv was injected onto both reference flow cell 1 and Siglec 15-Fc captured flow cell 2 for 150 seconds at flow rate of 30 ul/min followed by wash for 300 seconds. The flow cells were then regenerated with Glycine pH 2 buffer (GE) for 30 seconds at flow rate of 30 ul/mins. 8 concentration points from 300-0 nM was assayed per anti-Siglec15 scFv in a 96 well plate. The kinetics of scFvs binding to Siglec 15 protein was analyzed with Biacore T200 evaluation software version 3.0. The specific binding response unit was derived from subtraction of binding to reference flow cell 1 from Siglec15 captured flow cell 2. The Kon, Koff and KD values of exemplary anti-Siglec15 scFv antibodies were calculated and provided in Table 3 below.

TABLE 3
Kinetic Siglec15 Binding Features of
Exemplary Anti-Siglec15 Antibodies

| Exemplary Clones | Ka (1/ms) | Kd (1/s) | KD (M) |
|---|---|---|---|
| 2019EP47-A02 | 2.871E+4 | 0.001487 | 5.876E−8 |
| 2019EP47-A05 | 2.788E+4 | 7.771E−5 | 2.788E−9 |
| 2020EP032-A08 | 4.645E+4 | 8.422E−4 | 1.813E−8 |
| 2019EP47-A10 | 1.815E+4 | 6.023E−5 | 3.319E−9 |
| 2020EP032-A12 | 1.298E+5 | 2.936E−4 | 2.262E−9 |
| 2020EP032-B03 | 1.338E+5 | 2.820E−4 | 2.108E−9 |
| 2020EP032-C09 | 6.759E+4 | 0.001695 | 2.508E−8 |
| 2019EP47-C12 | ND | ND | Not fitting |
| 2020EP085-G5 | 1.318E+5 | 0.001442 | 1.094E−8 |
| 2020EP083-G11 | 3.184E+5 | 3.555E−4 | 1.117E−9 |
| 2020EP083-H01 | 1.426E+5 | 0.001487 | 1.043E−8 |
| 2020EP032-H11 | 5.668E+5 | 3.156E−4 | 5.567E−10 |

ND: not determined.

### ScFv Antibody Binding to Cell Surface Siglec15 Determined by FACS

To determine whether anti-Siglec15 scFvs bind to Siglec15 expressing cells, 200 nM of purified anti-Siglec 15 scFv antibodies were diluted in full medium and incubated with Siglec15/HEK293 and HEK293 cells in 96 wells plate on ice for 1 hour. Cells were spun down at 1200 rpm for 5 minutes at 4° C. to remove primary antibodies. Cells were then washed once with 200 uL of full medium per well. Samples were detected with premixed anti-His Biotin Streptavidin Alexa fluor 647 by adding 100 uL of diluted secondary antibody and incubated at 4° C. for 30 minutes in the dark. Samples were spun down at 1200 rpm for 5 minutes at 4° C. and washed twice with 200 uL of 1×PBS per well. Samples were then reconstituted in 200 uL of 1×PBS and read on Attune NxT cytometer. Analysis was performed using Attune NxT software plotting the overlaying the histogram of anti-Siglec15 scFvs binding onto both negative and target cell lines. The cell surface Siglec15 binding EC50 values for exemplary anti-Siglec15 scFv antibodies were calculated and shown in Table 4.

TABLE 4
EC50 Values of Exemplary Anti-Siglec15 Antibodies
for Binding to Cell Surface Siglec15

| Exemplary Clones | EC50 (nM) Siglec15/HEK293 |
|---|---|
| 2019EP47-A02 | 10.61 |
| 2019EP47-A05 | 88.34 |
| 2020EP032-A08 | 1.60 |
| 2019EP47-A10 | 111.7 |
| 2020EP032-A12 | 0.86 |
| 2020EP032-B03 | 1.22 |
| 2020EP032-C09 | 0.65 |
| 2019EP47-C12 | 13.09 |
| 2020EP085-G5 | 1.00 |
| 2020EP083-G11 | 0.87 |
| 2020EP083-H01 | 0.23 |
| 2020EP032-H11 | 10.61 |

In sum, a number of anti-Siglec15 antibodies were identified in this Example. Such antibodies show high binding affinity to human Siglec15, including cell surface Siglec15.

### Example 2. IgG Antibody Production and Characterization

This example describes production of exemplary anti-Siglec15 antibodies in IgG form (including 2019EP47-A02, 2019EP47-A05, 2019EP47-A10, 2019EP47-C12, 2020EP032-A08, 2020EP032-A12, 2020EP032-B03, 2020EP032-H11, 2020EP032-C09, 2020EP083-G11, 2020EP083-H01, and 2020EP085-G5) in a mammalian host cell and characterization of the IgG antibody thus produced.

Expression of IgG Antibody in Mammalian Host cells

Exemplary anti-Siglec15 monoclonal antibody was expressed transiently in ExpiHEK293-F cells in free style system (Invitrogen) according to standard protocol with a ratio of the plasmid DNA of heavy chain and light chain of 1:2. The cells were grown for five days before harvesting. The supernatant was collected by centrifugation and filtered through a 0.2 μm PES membrane. The antibody was purified by MabSelect PrismA protein A resin (GE Health). The protein was eluted with 100 mM Gly pH 2.5+150 mM NaCl and quickly neutralized with 20 mM citrate pH 5.0+300 mM NaCl. The antibody was then further purified by a Superdex 200 16/600 column. The monomeric peak fractions were pooled and concentrated. The final purified protein had an endotoxin of lower than 10 EU/mg and kept in 20 mM Histidine pH 6.0+150 mM NaCl.

### Anti-Siglec15 IgG Antibody Binding to Siglec15 in ELISA

An ELISA assay was developed to determine the EC50 of anti-Siglec15 IgG antibodies. Briefly, a 384 well plate was immobilized with human Siglec 15-HIS tagged recombinant protein at a final concentration of 2 ug/mL in 1×PBS in total volume of 25 uL per well. The plate was incubated overnight at 4° C. followed by blocking with 80 uL of superblock per well for 1 hour. Titration of purified anti-Siglec15 IgG was performed, starting at 200 nM 2-fold serial dilution, then 25 uL was added to the human Siglec15 immobilized wells and incubated for 1 hour with shaking. The Siglec15 binding was detected by adding 25 uL of anti-hFc HRP diluted at 1:5000 in 1×PBST. In between each step, the plate was washed 3 times with 1×PBST in a plate washer. The plate was then developed with 20 ul of TMB substrate for 5 mins and stopped by adding 20 ul of 2N sulfuric acid. The plate was read at OD450 nm Biotek plate reader and then plotted in Prism 8.1 software. Similar binding experiments were done for mouse Siglec15 and cyno Siglec15 to check the cross activity of the IgG antibodies to those two species.

Table 5 below shows the EC50 values of exemplary anti-Siglec15 IgG antibodies to human, mouse and cyno Siglec 15 determined by ELISA.

TABLE 5
Binding of IgG Antibodies to Siglec15 of Various Species

| | 2019EP47-A02 | 2019EP47-A10 | 2020EP32-B03 | 2020EP032-H11 |
|---|---|---|---|---|
| | EC50 (nM) | EC50 (nM) | EC50 (nM) | EC50 (nM) |
| rhSiglec15 His | 0.02893 | 0.1188 | 0.5271 | 0.02279 |
| rcynoSiglec15 His | — | 0.1709 | 1.2116 | 0.02576 |
| rmouseSiglec15 His | — | 0.1089 | 0.2477 | 0.01052 |

### Binding Kinetics of Anti-Siglec15 IgG Antibody to Siglec15 in SPR

Kinetic analysis of anti-Siglec15 IgG was assessed by SPR technology with Biacore T200. The assay was run with Biacore T200 control software version 2.0. For each cycle, 1 ug/mL of anti-Siglec15 IgG was captured for 60 seconds at flow rate of 10 ul/min on flow cell 2 in 1× HBSP buffer on Protein A sensor chip. 2-fold serial human Siglec15-HIS tagged protein was injected onto both reference flow cell 1 and anti-Siglec15 IgG captured flow cell 2 for 150 seconds at flow rate of 30 ul/mins followed by wash for 300 seconds. The flow cells were then regenerated with Glycine pH2 for 60 seconds at flow rate of 30 ul/mins. 8 concentration points from 100-0 nM were assayed per anti-Siglec15 IgG in a 96 well plate. The kinetics of Anti-Siglec15 IgG binding to Siglec 15 protein was analyzed with Biacore T200 evaluation software version 3.0. The specific binding response unit was derived from subtraction of binding to reference flow cell 1 from antibody captured flow cell 2. Table 6 shows the binding kinetics of the anti-Siglec15 IgG antibody as determined by SPR.

TABLE 6
Kinetic Siglec15 Binding Features of Exemplary
Anti-Siglec15 IgG Antibodies

| Exemplary Clones | Ka (1/ms) | Kd (1/s) | KD (M) |
|---|---|---|---|
| 2019EP47-A02 | 1.292E+5 | 3.112E−4 | 2.4 |
| 2019EP47-A10 | 9.644E+4 | 3.678E−4 | 3.8 |
| 2020EP032-H11 | NA* | NA* | 0.84 |

NA*: Kinetics did not fit. KD measured by steady kinetics fitting.

### Anti-Siglec15 IgG Antibody Binding to Cell Surface Siglec15 by FACS

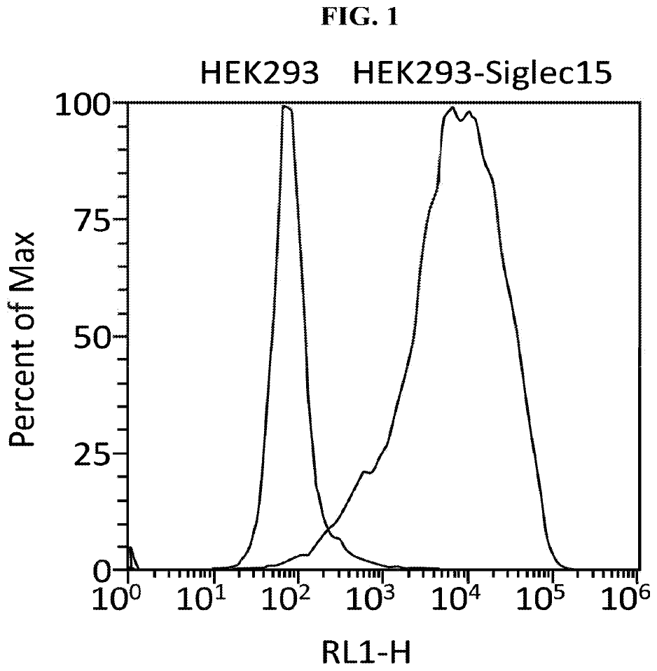

200 nM of purified anti-Siglec15 IgG antibodies were diluted in full medium and incubated with Siglec15/K562 and K562 cells in 96 wells plate on ice for 1 hour. Cells were spun down at 1200 rpm for 5 minutes at 4° C. to remove primary antibodies. Cells were then washed once with 200 uL of full medium per well. Samples were detected with anti-hFc Alexa fluor 647 by adding 100 uL of diluted secondary antibody and incubated at 4° C. for 30 minutes in the dark. Samples were spun down at 1200 rpm for 5 minutes at 4° C. and washed twice with 200 uL of 1×PBS per well. Samples were reconstituted in 200 uL of 1×PBS and read on Attune NxT cytometer. Analysis was done using Attune NxT software by plotting the overlay histogram of BCMA proteins binding onto both negative and target cell lines. Table 7 shows the binding activities of anti-Siglec15 Antibodies to Siglec15/HEK293 cells by FACS. See also FIG. 1, which shows the RL1-H plotted against Percent of Max for HEK293 and HEK293-Siglec 15 cells.

TABLE 7
EC50 Values of Exemplary Anti-Siglec15 IgG Antibodies
for Binding to Cell Surface Siglec15

| Exemplary Clones | Siglec15/HEK293 FACS Binding EC50 (nM) |
|---|---|
| 2019EP47-A02 | 0.35 |
| 2019EP47-A10 | Weak binding |
| 2020EP032-B03 | 1.92 |
| 2020EP032-H11 | 0.022 |

### Example 3. Anti-Siglec15 Antibody Competition with Reference Antibody 5G12 in SPR

5G12 IgG antibody is a mouse antibody capable of binding to human Siglec15 either purchased (Creative Biolabs, CAT #: HPAB-N0237-YC) or produced in-house. This anti-Siglec15 antibody was used as a reference antibody in the competition assay disclosed herein.

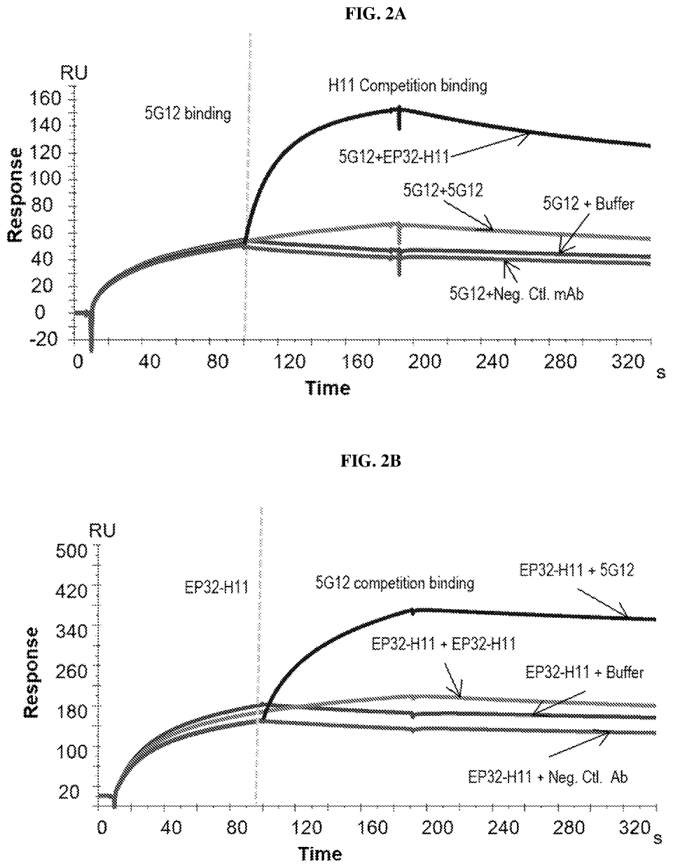

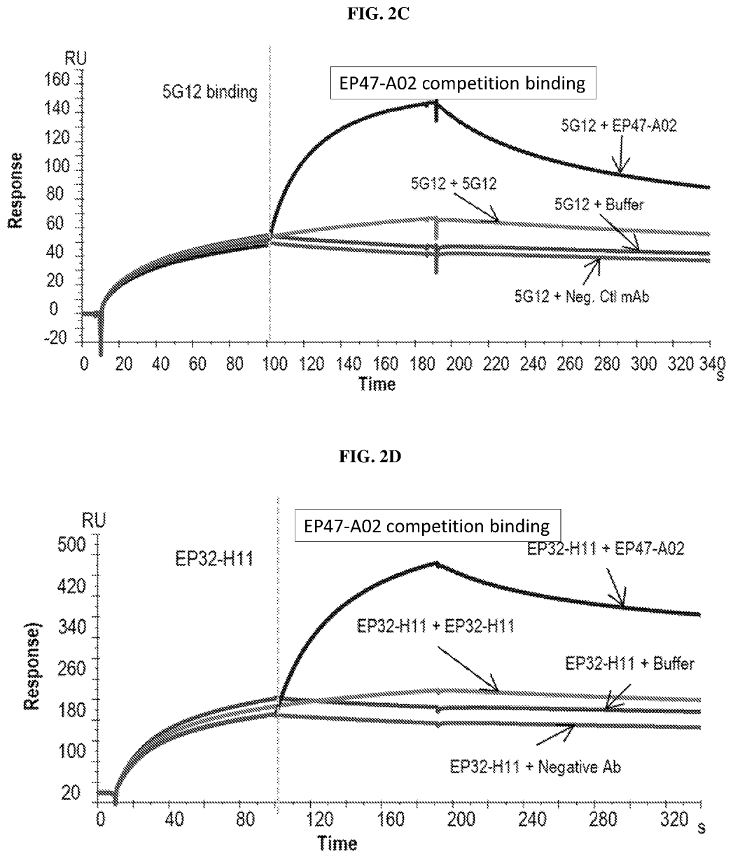

To evaluate whether exemplary anti-Siglec15 antibodies disclosed herein bind to an overlapping or distinct epitope relative to 5G12 IgG, an epitope binning assay was developed with Biacore T200. Briefly, human Siglec 15-HIS tag protein was immobilized on the CM5 sensor chip FC2 at 300 RU level. In the first assay format, 5G12 at 300 nM was injected to FC1 and FC2 for 90 sec at flow rate of 30 ul/min to reach binding saturation, followed by injection of 300 nM of 5G12, or anti-Siglec15 2020EP32-H11 or 2019EP47-A02 or negative IgG for 90 sec at the same flow rate. In the second format, 2020EP32-H11 at 300 nM was injected to FC1 and FC2 for 90 sec at flow rate of 30 ul/min to reach binding saturation, followed by injection of 300 nM of 2020EP32-H11, or 5G12, or anti-Siglec 15 2019EP47-A02 or negative IgG for 90 sec at the same flow rate. The data was analyzed with Biacore T200 evaluation software version 3.0. The dual baseline was set for the analysis. The binding response unit was calculated from subtraction of FC1 from FC2. FIGS. 2A and 2B show the competition between 2020EP32-H11 and 5G12 and FIGS. 2C and 2D show the competition between A02 and 5G12 competition in SPR analysis.

### Example 4. Anti-Siglec15 Antibody Binding Specificity

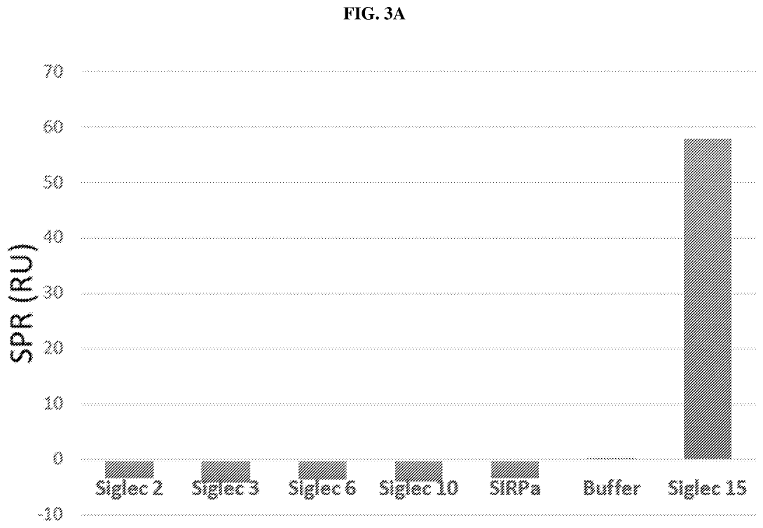

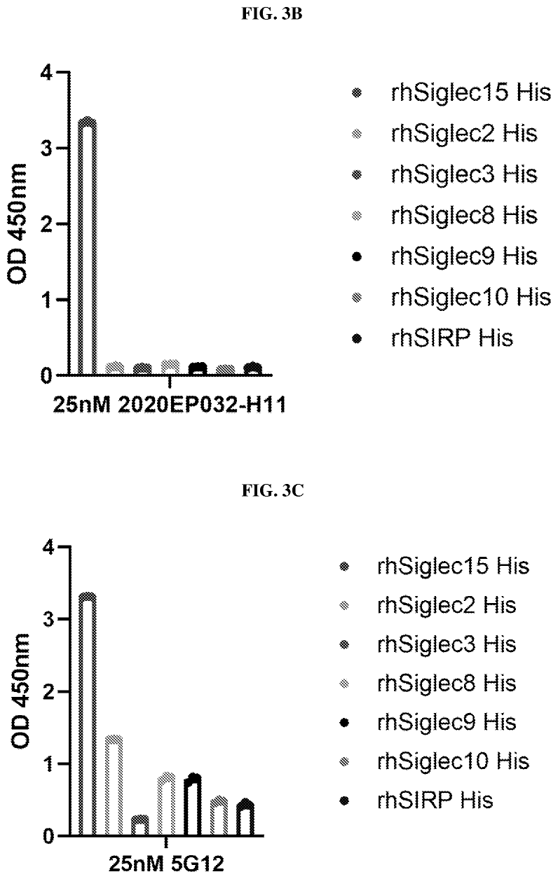

To test the Anti-Siglec15 IgG selectivity to Siglec family proteins, an SPR assay was developed with Biacore T200. Briefly, anti-Siglec15 IgG at 1 ug/mL concentration in HBSP+ buffer was captured on Protein A sensor chip on FC2 at 10 ul/min flow rate for 60 sec. Human Siglec 15-HIS, Siglec 2-HIS, Siglec 3-HIS, Siglec 6-HIS, Siglec 10-HIS, and SIRPα-HIS tagged protein at 300 nM were injected onto both FC1 control and FC2 with captured anti-Siglec15 IgG for 90 secs at flow rate of 30 ul/mins. The binding response unit was calculated by subtraction of FC1 from FC2 and analyzed with Biacore T 200 evaluation software version 3.0. FIG. 3A shows the binding activities of anti-Siglec15 antibody 2020EP32-H11 to selected siglec family proteins and macrophage expression SIRPα (Signal Regulatory Protein Alpha) protein.

An ELISA assay was developed to determine the selectivity binding of anti-Siglec 15 IgG antibodies to different Siglec family proteins. Briefly, a 384 well plate was immobilized with human Siglec 15-HIS, human Siglec 2-HIS, human Siglec 3-HIS, human Siglec 8-HIS, human Siglec 9-HIS, human Siglec 10-HIS, and human SIRPα-HIS tagged recombinant proteins at final concentration of 2 ug/mL in 1×PBS in total volume of 25 uL per well. The plate was incubated overnight at 4° C. followed by blocking with 80 uL of superblock per well for 1 hour. 25 uL of anti-Siglec 15 IgG at 25 nM was added to each different Siglec family recombinant protein immobilized wells and incubated for 1 hour with shaking. The Siglec15 binding was detected by adding 25 uL of anti-hFc HRP diluted at 1:10,000 in 1×PBST. In between each step, the plate was washed 3 times with 1×PBST in a plate washer. The plate was then developed with 20 ul of TMB substrate for 5 mins and stopped by adding 20 ul of 2 N sulfuric acid. The plate was read at OD450 nm Biotek plate reader and then plotted in Prism 8.1 software. FIGS. 3B-3C show the binding activities of anti-Siglec15 antibodies 2020EP32-H11 and 5G12 to selected siglec family proteins and macrophage expression SIRPα protein, respectively.

### Example 5. Anti-Siglec15 Antibody Immune Cell Activity

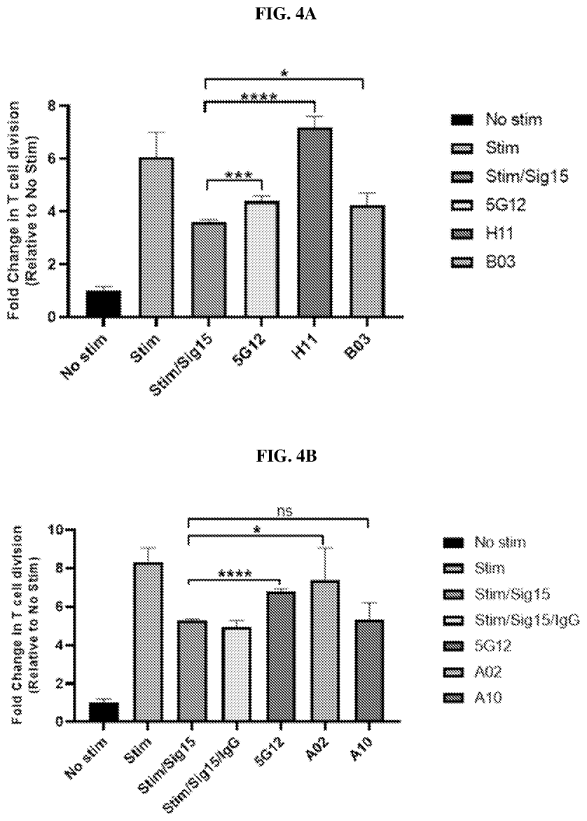

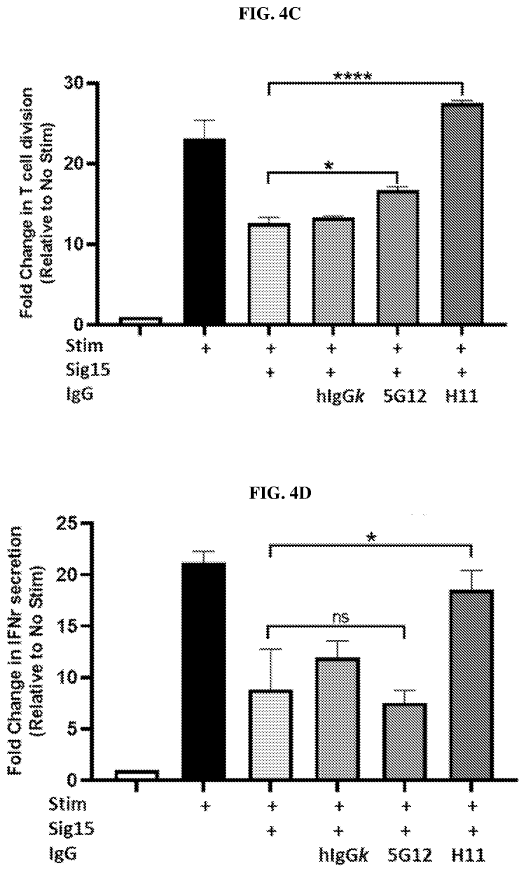

Freshly thawed human PBMC were pre-incubated in CellTrace CFSE for 15 minutes according to the manufacturer's protocol. The incubation was then blocked with a 5× volume of complete media, and PBMCs were washed twice. The CFSE stained PBMCs were plated in a 96 well plate at 100,00 cells per well in media containing 1 nM IL2 and 2.5 uL per well of Immunocult Human CD3/CD28 T Cell activator. Recombinant Human Siglec 15 and anti-Siglec 15 antibodies were added to wells as appropriate at final concentrations of 176 nM and 80 nM respectively. Cells were incubated at 37° C. with 5% CO2 for 4 days. Cells were stained for Viability and CD3, and the proliferation of the Live, CD3+ cell population was quantified by observing the CFSE signal on an Attune NXT flow cytometer. FIGS. 4A-4B show the single concentration screening of IgG antibodies to human health donor PBMC T cell activation.

Freshly thawed human PBMC were pre-incubated in CellTrace CFSE for 15 minutes according to the manufacturers protocol. The incubation was then blocked with a 5× volume of complete media, and PBMCs were washed twice. The CFSE stained PBMCs were plated in a 96 well plate at 10,000 cells per well in media containing 1 nM IL2 and 2.5 uL per well of Immunocult Human CD3/CD28 T Cell activator. Recombinant Human Siglec 15 and anti-Siglec 15 antibodies were added to wells as appropriate at final concentrations of 176 nM and 80 nM respectively. Cells were incubated at 37° C. with 5% CO2 for 4 days. Media was collected for detection of IFNγ secretion by ELISA. Cells were stained for Viability and CD3, and the proliferation of the Live, CD3+ cell population was quantified by observing the CFSE signal on an Attune NXT flow cytometer.

IFNγ secretion was measured by DuoSet ELISA following manufacturer's instructions, briefly: immunosorbent plates were coated overnight with IFNγ detection antibodies. The following day, plates were washed with 1× TBS-T and blocked. After a further wash, the media, diluted in PBS, was then added to appropriate wells. After incubation, plates were washed, and incubated with the supplied IFNγ detection antibody. The ELISAs were developed with an HRP secondary antibody and TMB substrate, with the reaction stopped with 2N sulfuric acid. IFNγ concentrations in the media was quantified by comparing the optical density at 450 nm on a microplate reader of the samples with a standard curve generated with known concentrations of the cytokine. FIGS. 4C-4D compare the 2020EP32-H11 IgG antibody with 5G12 in T cell activation.

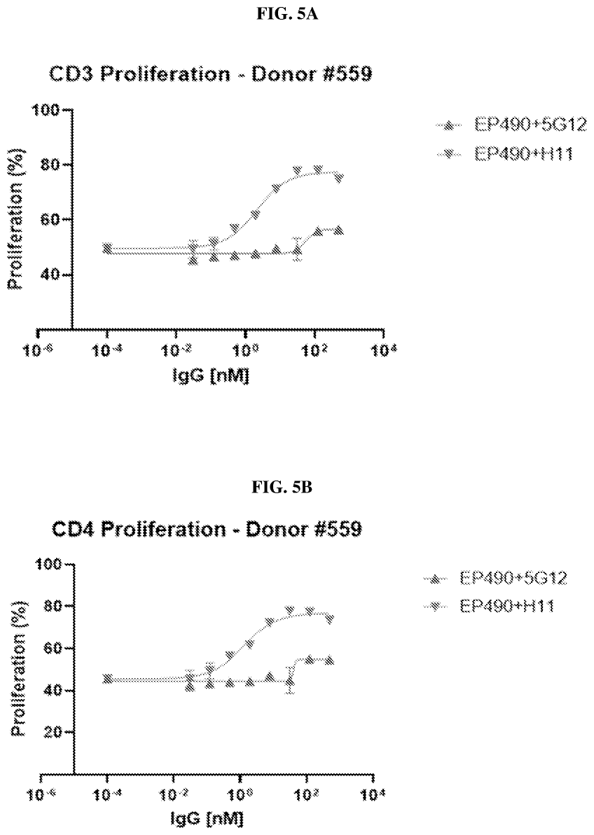

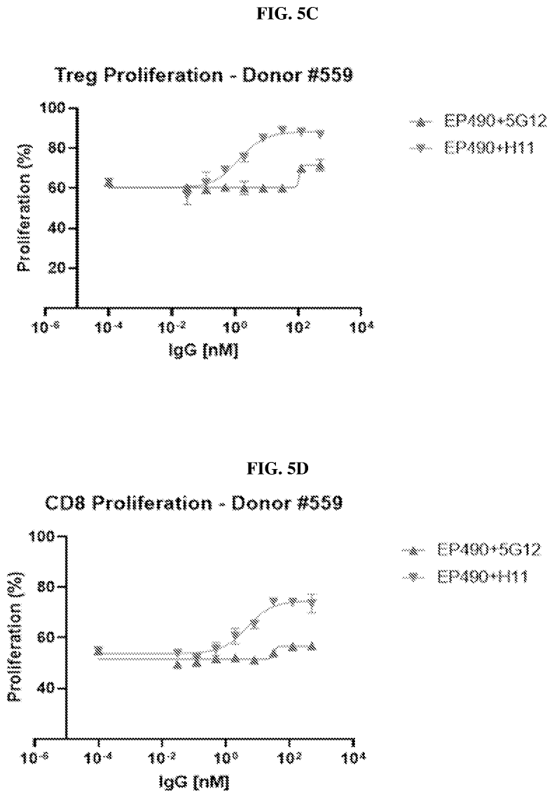

Human PBMCs from donor #559 were thawed and stained with CellTrace CFSE following the manufacturer's instructions. CFSE stained cells were plated in a 96 well plate, 100,000 cells per well, in media containing: 2.9 nM recombinant human Siglec15, 1 nM IL2, and 25 uL/mL of Immunocult Human CD3/CD28 T Cell activator. A titration of anti-Siglec 15 antibodies (5G12 or 2020EP32-H11) was added to respective wells, starting at 500 nM. Cells were incubated at 37° C. with 5% CO2 for 7 days. After incubation, cells were stained for Viability, CD3, CD4, CD8, and FOXP3. Proliferation was measured by observing the CFSE signal on an Attune NXT flow cytometer within the CD3, CD4, CD8 and TReg populations. FIGS. 5A-5D show the dose dependent T cell activation and subtypes of T cells in donor #559 PBMC.

Human PBMCs from donor #622 were thawed and stained with CellTrace CFSE following the manufacturer's instructions. CFSE stained cells were plated in a 96 well plate, 100,000 cells per well, in media containing: 2.9 nM recombinant human Siglec15, 1 nM IL2, and 25 uL/mL of Immunocult Human CD3/CD28 T Cell activator. A titration of anti-Siglec 15 antibodies (5G12 or H11) was added to respective wells, starting at 500 nM. Cells were incubated at 37° C. with 5% CO2 for 7 days. After incubation, cells were stained for Viability, CD3, CD4, CD8, and FOXP3. Proliferation was measured by observing the CFSE signal on an Attune NXT flow cytometer within the CD3, CD4, CD8 and TReg populations.

Human PBMCs from donors #559 and #622 were thawed and stained with CellTrace CFSE following the manufacturer's instructions. CFSE stained cells were plated in a 96 well plate, 100,000 cells per well, in media containing: 2.9 nM recombinant human Siglec15, 1 nM IL2, and 25 uL/mL of Immunocult Human CD3/CD28 T Cell activator. A titration of anti-Siglec 15 antibodies (5G12 or 2020EP32-H11) was added to respective wells, starting at 500 nM. Cells were incubated at 37° C. with 5% CO2 for 7 days. After incubation, media was collected from each well for cytokine analysis. Cells were stained for Viability, CD3, and CD56. Proliferation was measured by observing the CFSE signal on an Attune NXT flow cytometer within the NK cell population.

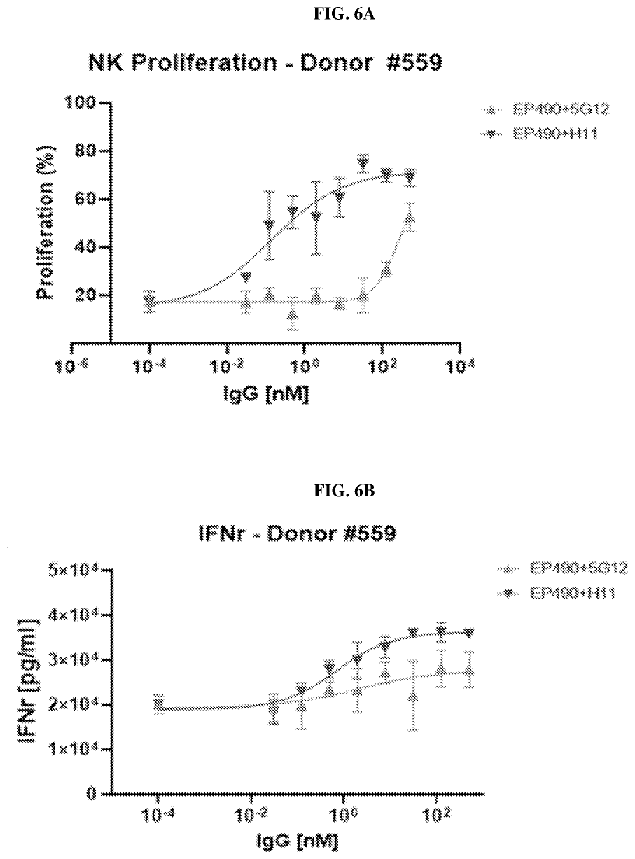

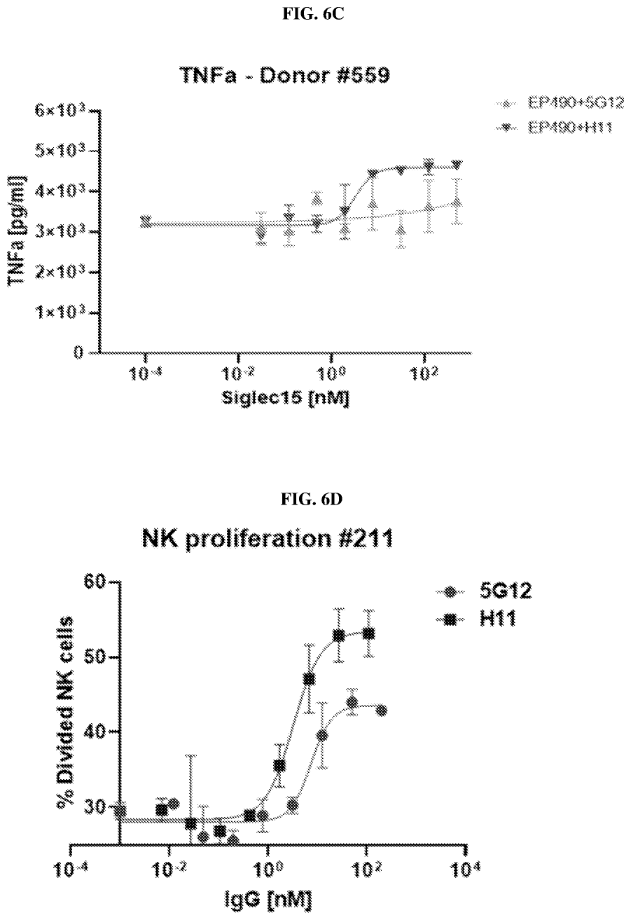

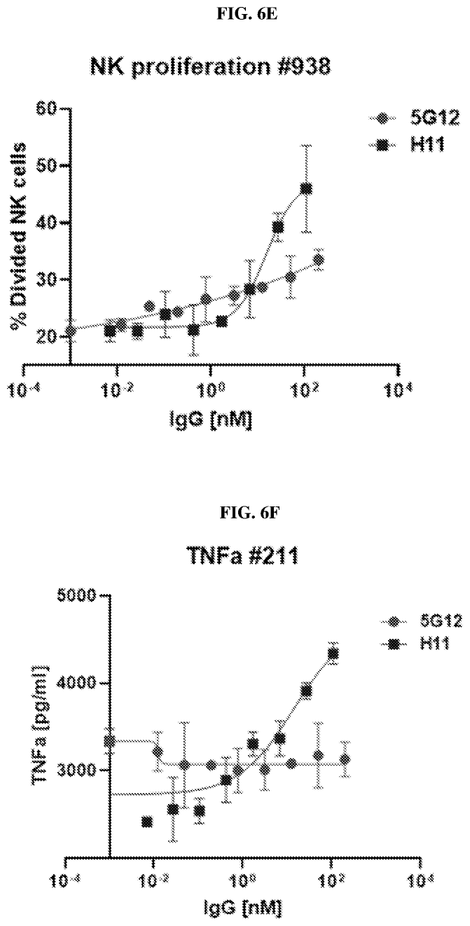

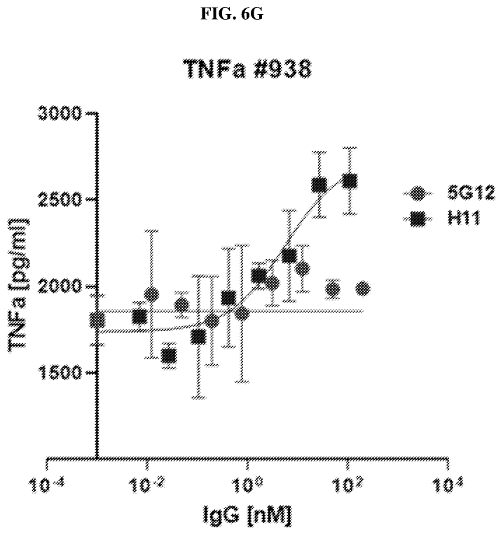

IFNγ and TNFα secretion was measured by DuoSet ELISA following manufacturer's instructions, briefly: immunosorbent plates were coated overnight with relevant detection antibodies. The following day, plates were washed with 1× TBS-T and blocked. After a further wash, the media, diluted in PBS, was added to appropriate wells. After incubation, plates were washed, and incubated with the supplied detection antibody. The ELISAs were developed with an HRP secondary antibody and TMB substrate, with the reaction stopped with 2N sulfuric acid. IFNγ and TNFα concentrations in the media was quantified by comparing the optical density at 450 nm on a microplate reader of the samples with a standard curve generated with known concentrations of the cytokine. FIGS. 6A-6C show the dose dependency in NK cell activation with donor #559.

Human PBMCs from donors #211 and #938 were thawed and stained with CellTrace CFSE following the manufacturer's instructions. CFSE stained cells were plated in a 96 well plate, 100,000 cells per well, in media containing: 2.9 nM recombinant human Siglec15, 1 nM IL2, and 25 uL/mL of Immunocult Human CD3/CD28 T Cell activator. A titration of anti-Siglec 15 antibodies (5G12 or 2020EP32-H11) was added to respective wells, starting at 500 nM. Cells were incubated at 37° C. with 5% CO2 for 7 days. After incubation, media was collected from each well for cytokine analysis. Cells were stained for Viability, CD3, and CD56. Proliferation was measured by observing the CFSE signal on an Attune NXT flow cytometer within the NK cell population.

IFNγ and TNFα secretion was measured by DuoSet ELISA following manufacturer's instructions, briefly: immunosorbent plates were coated overnight with relevant detection antibodies. The following day, plates were washed with 1× TBS-T and blocked. After a further wash, the media, diluted in PBS, was added to appropriate wells. After incubation, plates were washed, and incubated with the supplied detection antibody. The ELISAs were developed with an HRP secondary antibody and TMB substrate, with the reaction stopped with 2N sulfuric acid. IFNγ and TNFα concentrations in the media was quantified by comparing the optical density at 450 nm on a microplate reader of the samples with a standard curve generated with known concentrations of the cytokine. FIGS. 6D-6G show dose dependency in NK cell activation with donor #211 and #938.

In sum, the anti-Siglec 15 antibodies tested in this example showed immune activation activity. Certain clones (e.g., 2020EP32-H11) exhibited higher immune activation activity relative to control antibody 5G12.

### Example 6. ADCC Activities of Exemplary Anti-Siglec15 Antibodies

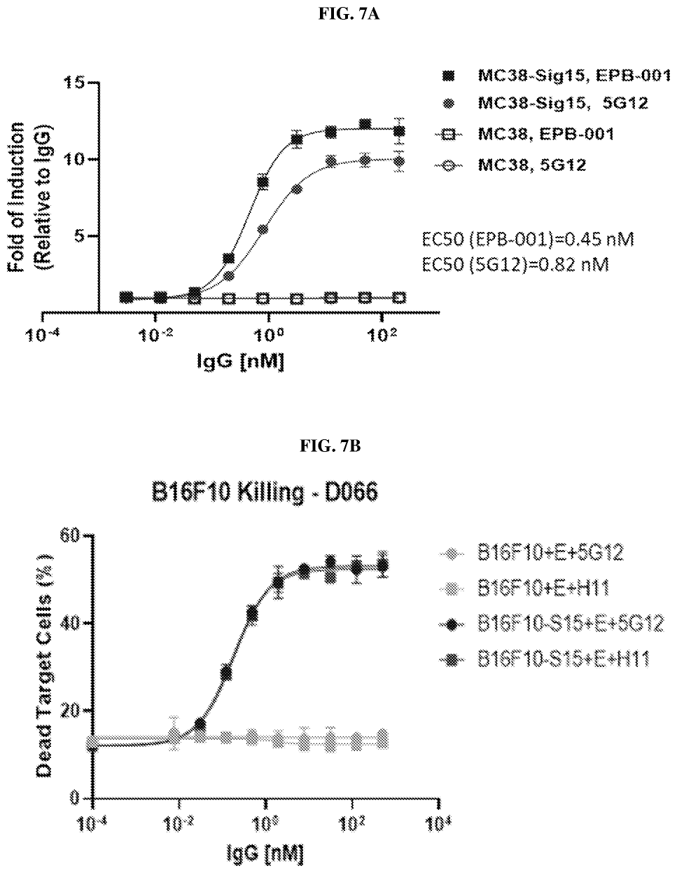

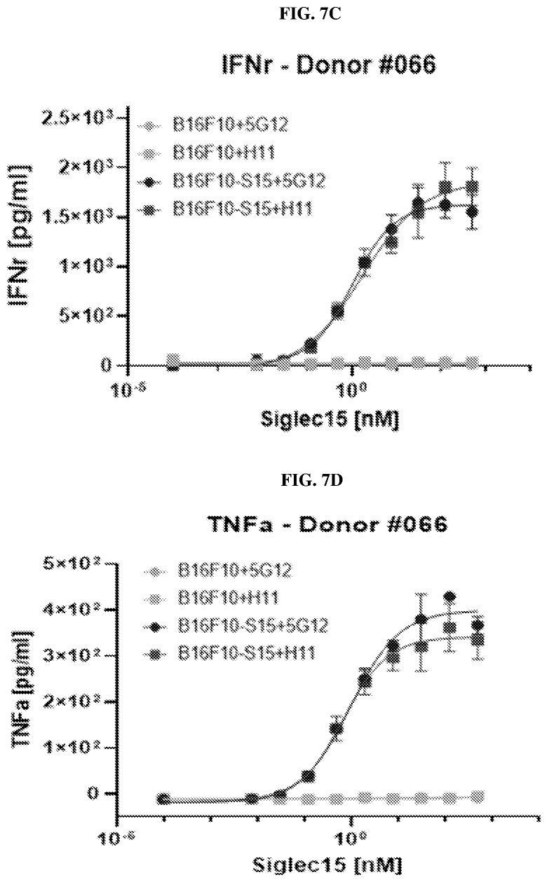

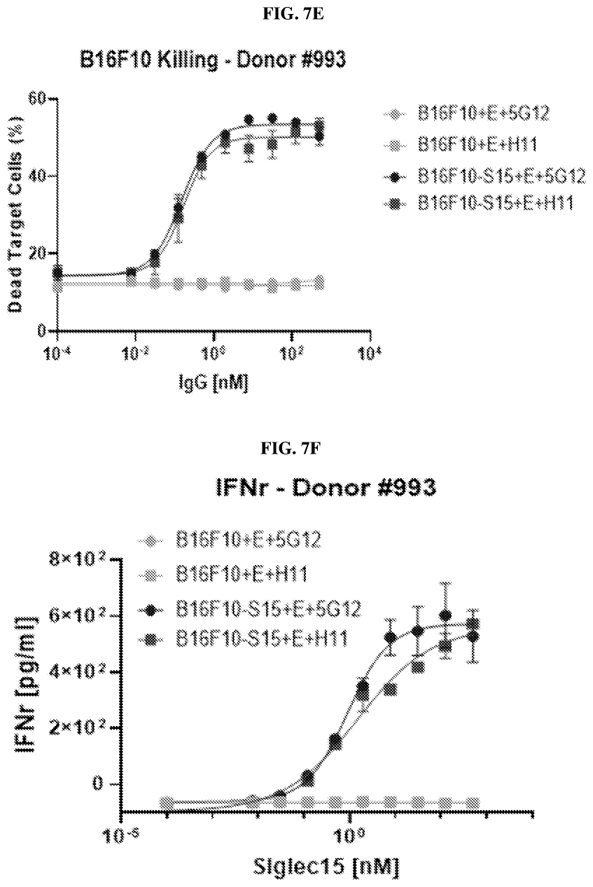

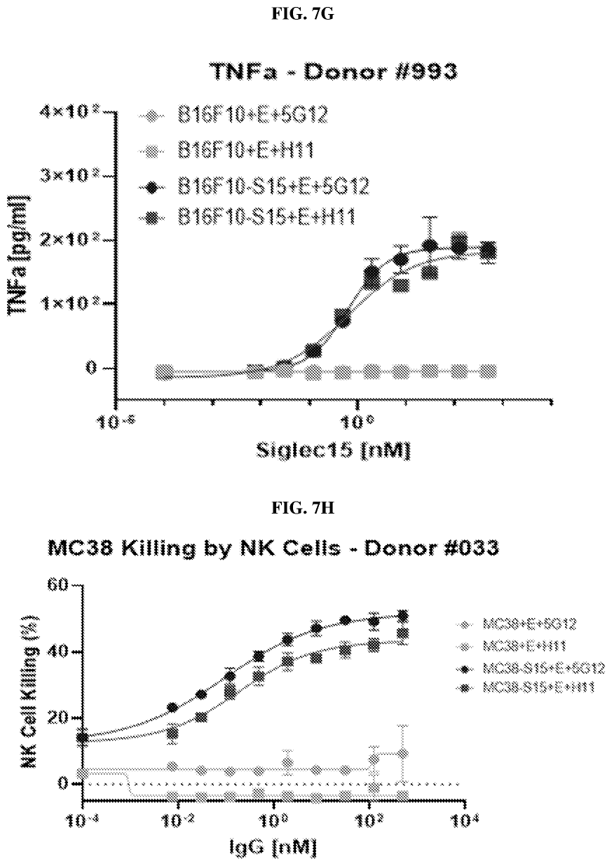

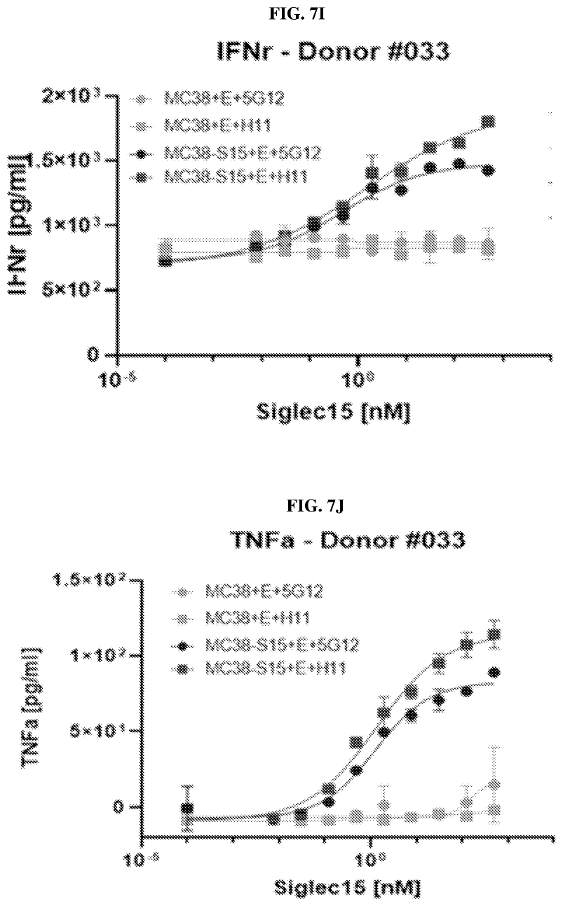

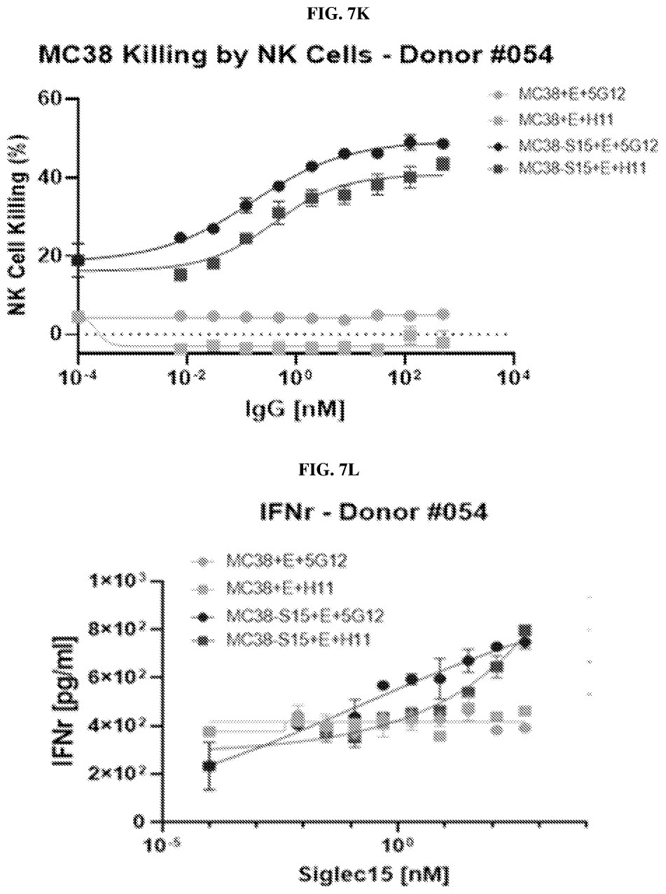

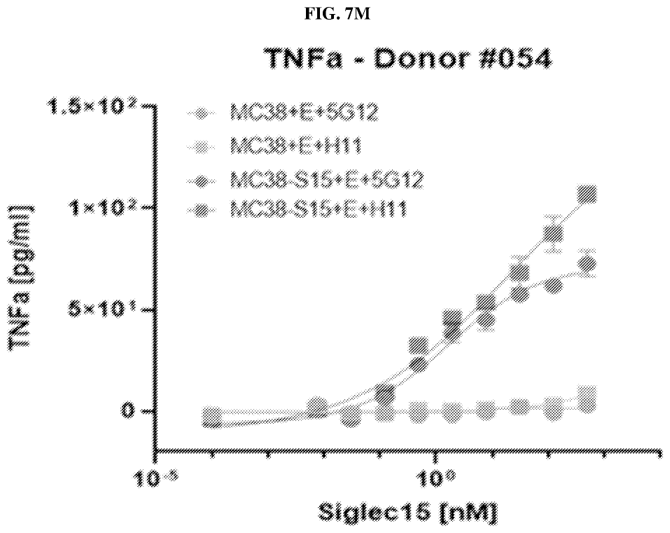

The ADCC effect of anti-Siglec15 antibodies was assessed using the Promega ADCC Reporter Bioassay Kit, following the manufacturer's instructions. Briefly: MC38 and MC38-Siglec15 target cells were seeded in a 96 well white plate at 10,000 cells per well in complete media and left overnight at 37° C. The following day, the media was removed and replaced with ADCC buffer containing a titration of anti-Siglec 15 antibodies (5G12 and 2020EP32-H11) starting at 200 nM. Effector Jurkat NFAT Luciferase cells were added at 37500 cells per well. Cells were left at 37° C. in 5% CO2 for 24 hours. The following day, plates were incubated at room temperature for 15 minutes and then 75 uL of Bioglo Luciferase was added to appropriate wells. After 30 minutes, luciferase signal was quantified by a microplate reader detecting absorbance at 450 nm. Fold induction of ADCC was calculated by normalizing the luminescence signal to the no drug control well. FIGS. 7A-7B show the ADCC effects of antibodies to MC38-hSiglec 15 cell line by Jurkat NFAT Luciferase assay.

B16F10 and B16F10-Siglec15 cells were stained with CellTrace FarRed according to the manufacturer's instructions. CellTrace stained cells were seeded in 96 well plates at 100,000 cells per well. Freshly isolated NK cells from donors: 066 and 993 were added to respective wells at a concentration of 100,000 cells per well. A titration of anti-Siglec15 antibodies (5G12 and 2020EP32-H11) was added to respective wells starting at 500 nM. IL2 was added to each well at a final concentration of 1 nM. Cells were incubated at 37° C. with 5% CO2 for 4 hours. After incubation, cells were washed with PBS, and stained with a 1:800 dilution of Zombie Aqua fixable viability dye from Biolegend. After further washing, cells were fixed with 4% PFA. Samples were analyzed using an Attune NXT Flow Cytometer, with the percentage of dead target cells quantified relative to the total number of target cells observed. FIGS. 7C-7H shows the ADCC effects of the tested antibodies to B16F10-hSiglec15 cell line by NK cells.

MC38 and MC38-Siglec 15 cells were stained with CellTrace FarRed according to the manufacturer's instructions. CellTrace stained cells were seeded in 96 well plates at 100,000 cells per well. Freshly isolated NK cells from donors: 033 and 054 were added to respective wells at a concentration of 100,000 cells per well. A titration of anti-Siglec15 antibodies (5G12 and 2020EP32-H11) was added to respective wells starting at 500 nM. IL2 was added to each well at a final concentration of 1 nM. Cells were incubated at 37° C. with 5% CO2 for 4 hours. After incubation, cells were washed with PBS, and stained with a 1:800 dilution of Zombie Aqua fixable viability dye from Biolegend. After further washing, cells were fixed with 4% PFA. Samples were analyzed using an Attune NXT Flow Cytometer, with the percentage of dead target cells quantified relative to the total number of target cells observed. FIG. 7I-7M shows the ADCC effects of the tested antibodies to MC38-hSiglec 15 cell line by NK cell.

In summary, exemplary clone 2020EP32-H11 induced ADCC effect when co-incubated with target cells expressing surface Siglec 15 and NK cells relative to control antibody 5G12, 2020EP32-H11 showed similar ADCC activity.

### Example 7. Pharmacodynamic Analysis of Anti-Siglec15 in B16F10-HSiglec15 Tumor Model

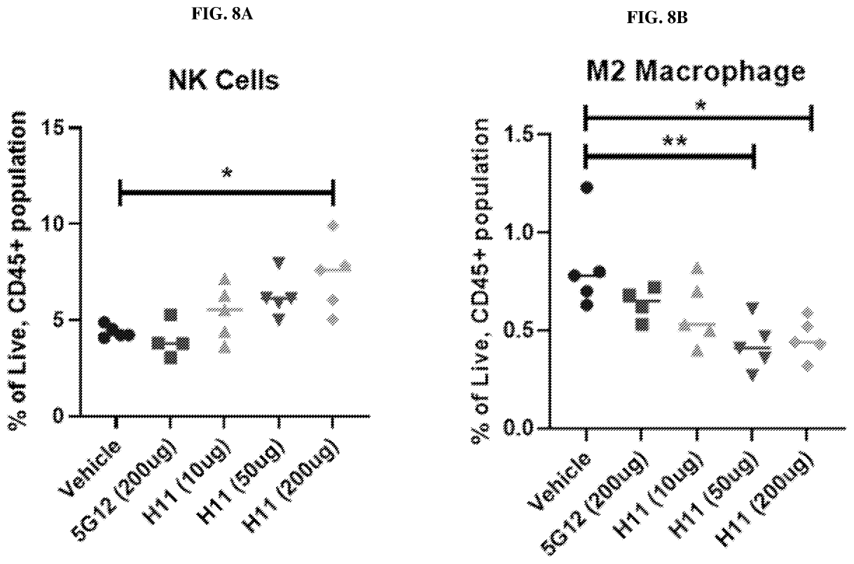

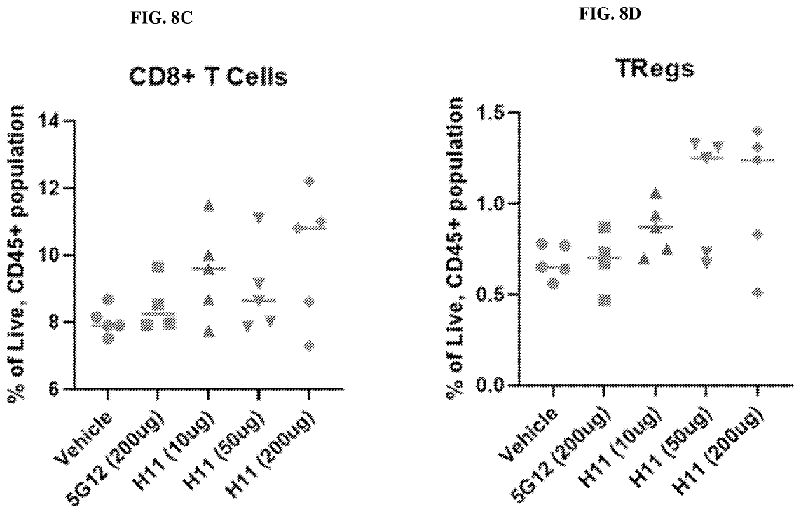

7 week old, female C57BL/6 mice were inoculated with 0.1×10⁶ B16F10-Siglec15 cells per mouse in 50% Matrigel subcutaneously into the flank. Tumors were allowed to grow until the mean tumor volume reached ˜80 mm³ when the mice were randomized and placed into treatment groups. Mice were dosed with either the vehicle, 5G12 (200 ug per dose), or H11 (10, 50, or 200 ug per dose). Mice were dosed every 4 days with 200 uL per dose. All doses were given IP. 13 days after the first dose, peripheral blood was drawn from the mice, and PBMCs were isolated. Cells were stained for their viability, and with antibodies for: CD45, CD3, NKp46, CD4, CD8, FOXP3, MHCII, and CD206. The percentage of the live, CD45+ population that were NK cells, M2 Macrophages, CD8+ T cells, and TRegs was quantified. FIGS. 8A-8D show the immune cell profiling in B16F10-hSiglec15 tumor bearing mice.

### Example 8: Pharmacokinetic (PK) Analysis of Exemplary Anti-Siglec15 Monoclonal Antibody H11 mAb in Cynomolgus Monkeys

2 to 5 years old male non-naïve Cynomolgus monkeys were used for pharmacokinetics studies of the anti-Siglec 15 antibodies disclosed herein, using H11 monoclonal antibody as an example.

In brief, monkeys were grouped into three groups with 2 monkeys in each group. The monkeys in each of the three groups were administered the antibody at 0 mg/kg, 5 mg/kg, or 20 mg/kg intravenously by bolus injection via the peripheral vein. Blood samples were collected from each group at the following time points: Pre-dose, 5 min, 1 hrs, 2 hrs, 4 hrs, 8 hrs, 12 hrs, 24 hrs (Day 2), 36 hrs, 48 hrs (Day 3), 72 hrs (Day 4), 96 hrs (Day 5), 120 hrs (Day 6), 144 hrs (Day 7), 168 hrs (Day 8), 192 hrs (Day 9), 216 hrs (Day 10), 240 hrs (Day 11), 264 hrs (Day 12), 288 hrs (Day 13), and 312 hrs (Day 14) post dose. The blood samples (˜0.5 ml) from each time point were collected from animals via peripheral vessel. The plasma samples were further prepared by centrifugation at 4° C., 3200 g for 10 minutes, and then quickly transferred to tubes and flash frozen over dry ice and kept at −60° C. for PK analysis.

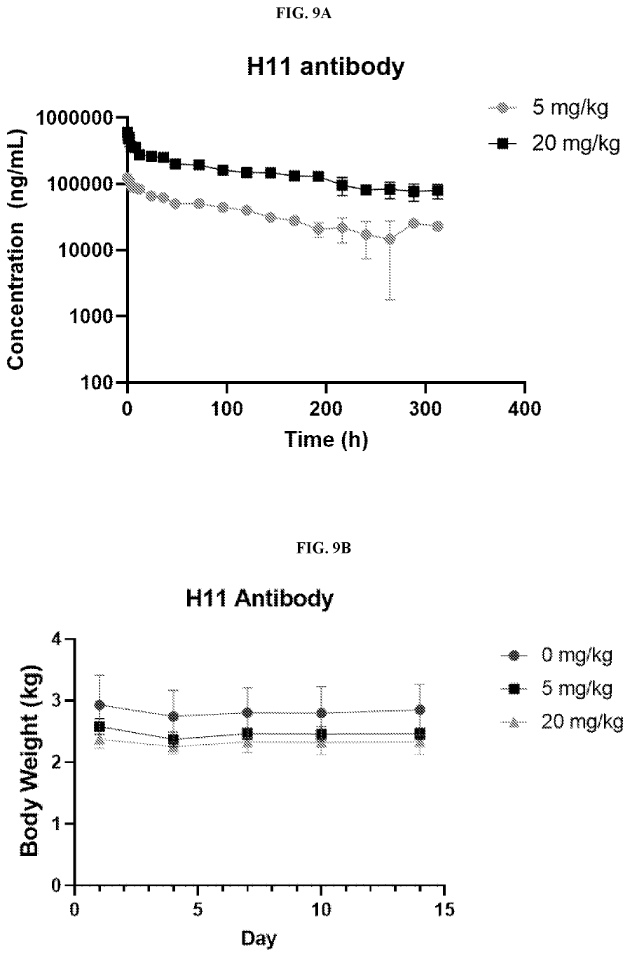

An ELISA assay was used to determine the concentration of the H11 antibody in monkey plasma using the plasma samples prepared as described above. The H11 antibody was captured by His-tag human Siglec-15 (Cat. No. SG5-H52H3-100 ug, Acro) and was detected by Goat Anti-Human IgG, Monkey ads-HRP (Cat. No. 2049-05, Southern Biotech.). A standard curve in the same format with purified H11 antibody was generated to measure the H11 concentrations in plasma. Plasma was diluted to appropriate volume so that the detected concentration of H11 fell into the linear range in the standard curve. Linear trapezoidal method in Phoenix WinNonlin 6.3 program was used to calculate the PK parameters shown in Table 8 below. H11 has a half-life of 123 hours at 5 mg/mg and 166 hours at 20 mg/kg. FIG. 9A shows the concentration of H11 in plasma over time and FIG. 9B shows the body weight change of the monkeys during the study.

TABLE 8
H11 mAb PK Analysis in Cyno Monkeys

| PK Parameters | 5.0 mg/kg | 20.0 mg/kg |
|---|---|---|
| T_{1/2} (h) | 123 | 166 |
| Vdₛₛ (L/kg) | 0.0598 | 0.0718 |
| Cl (mL/min/kg) | 0.00658 | 0.00512 |
| AUC₀₋ₗₐₛₜ (ng · h/mL) | 10924735 | 47013590 |

In sum, the pharmacokinetic studies reported in this example show that exemplary antibody H11 can maintain a suitable plasma concentration for as long as over 300 hours and no obvious toxicity was observed as evidenced by the no body weight loss in the monkeys treated by the antibody.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### Equivalents

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. An isolated antibody that binds sialic acid-binding Ig-like lectin 15 (Siglec15), wherein the antibody binds to the same epitope as a reference antibody or competes against the reference antibody from binding to Siglec-15, and wherein the reference antibody is selected from the group consisting of 2019EP47-A02, 2019EP47-A05, 2019EP47-A10, 2019EP47-C12, 2020EP032-A08, 2020EP032-A12, 2020EP032-B03, 2020EP032-H11, 2020EP032-C09, 2020EP083-G11, 2020EP083-H01, and 2020EP085-G5.

2. The isolated antibody of claim 1, wherein the antibody comprises:
(a) a heavy chain complementary determining region 1 (HC CDR1), a heavy chain complementary determining region 2 (HC CDR2), and a heavy chain complementary determining region 3 (HC CDR3), wherein the HC CDR1, HC CDR2, and HC CDR3 collectively are at least 80% identical to the heavy chain CDRs of the reference antibody; and/or
(b) a light chain complementary determining region 1 (LC CDR1), a light chain complementary determining region 2 (LC CDR2), and a light chain complementary determining region 3 (LC CDR3), wherein the LC CDR1, LC CDR2, and LC CDR3 collectively are at least 80% identical to the light chain CDRs of the reference antibody.

3. The isolated antibody of claim 1, wherein the HC CDRs of the antibody collectively contain no more than 8 amino acid residue variations as compared with the HC CDRs of the reference antibody; and/or wherein the LC CDRs of the antibody collectively contain no more than 8 amino acid residue variations as compared with the LC CDRs of the reference antibody.

4. The isolated antibody of claim 1, wherein the antibody comprises a V_{H} that is at least 85% identical to the V_{H} of the reference antibody, and/or a V_{L} that is at least 85% identical to the V_{L} of the reference antibody.

5. The isolated antibody of claim 1, wherein the antibody has a binding affinity of less than about 50 nM to Siglec15 expressed on cell surface, optionally wherein the binding affinity is less than 10 nM.

6. The isolated antibody of claim 5, wherein the antibody has a binding affinity of less than 5 nM, optionally 1.5 nM, to Siglec 15 expressed on cell surface.

7. The isolated antibody of claim 1, which comprises the same heavy chain complementary determining regions (HC CDRs) and the same light chain complementary determining regions (LC CDRs) as the reference antibody.

8. The isolated antibody of claim 7, which comprises the same V_{H} and the same V_{L} as the reference antibody.

9. The isolated antibody of claim 1, wherein the antibody is a human antibody or a humanized antibody.

10. The isolated antibody of claim 1, wherein the antibody is a full-length antibody or an antigen-binding fragment thereof.

11. The isolated antibody of claim 1, wherein the antibody is a single-chain antibody (scFv).

12. The isolated antibody of claim 11, wherein the antibody is a fusion polypeptide comprising the scFv.

13. A nucleic acid or a set of nucleic acids, which collectively encodes the antibody of claim 1.

14. The nucleic acid or the set of nucleic acids of claim 13, which is a vector or a set of vectors.

15. The nucleic acid or the set of nucleic acids of claim 14, wherein the vector is an expression vector.

16. A host cell comprising the nucleic acid or the set of nucleic acids of claim 13.

17. A pharmaceutical composition comprising the antibody of claim 1, the nucleic acid or nucleic acids encoding the antibody, or the host cell comprising the nucleic acid, and a pharmaceutically acceptable carrier.

18. A method for inhibiting Siglec15 or Siglec15⁺ cells in a subject, comprising administering to a subject in need thereof any effective amount of the pharmaceutical composition of claim 17.

19. The method of claim 18, wherein the subject is a human patient having Siglec-15⁺ pathogenic cells.

20. The method of claim 18, wherein the subject is a human patient having Siglec15+ disease cells, optionally wherein the disease cells are tumor cells or immune cells.

21. The method of claim 20, wherein the human patient has a Siglec 15+ cancer, which optionally is selected from the group consisting of non-small cell lung cancer (NSCLC), ovarian cancer, breast cancer, head-and-neck cancer, renal carcinoma, pancreatic cancer, endometrial cancer, urothelial cancer, thyroid cancer, colon cancer, colorectal cancer, melanoma, liver cancer, and gastric cancer.

22. A method for detecting presence of Siglec-15, comprising:
(i) contacting an antibody of claim 1 with a sample suspected of containing Siglec-15, and
(ii) detecting binding of the antibody to Siglec-15.

23. The method of claim 22, wherein the antibody is conjugated to a detectable label.

24. The method of claim 22, wherein the Siglec-15 is expressed on cell surface.

25. The method of claim 22, wherein the contacting step is performed by administering the antibody to a subject.

26. A method of producing an antibody binding to Siglec-15, comprising:
(i) culturing the host cell of claim 16 under conditions allowing for expression of the antibody that binds Siglec-15; and
(ii) harvesting the antibody thus produced from the cell culture.
